(19) United States
(12) Patent Application Publication
(10) Pub. No.: US 2004/0225252 A1
(43) Pub. Date: Nov. 11, 2004

(54) System and method for operating an infusion pump

(76) Inventors: John Gillespie JR., Libertyville, IL (US); Patrick D. Baird, Kenosha, WI (US); Patricia L. McNamara, Fox River Grove, IL (US); Ken Ganung, Bartlett, IL (US); Matthew Mayfield, Chicago, IL (US); Matthew Jordan, Chicago, IL (US)

Correspondence Address: Francis C. Kowalik, Esq., Corporate Counsel, Law Department, BAXTER INTERNATIONAL INC., One Baxter Parkway, DF3-2E, Deerfield, IL, 60015, US

(21) Appl. No.: 10/172,808

(22) Filed: Jun. 14, 2002

(51) Int. Cl. A61N 1/30; F04B 1/00

(52) U.S. Cl. 604/19; 417/53

(57) ABSTRACT

A method for operating an infusion pump is disclosed comprising: providing a pumping apparatus having an infusion pump and a processor operably coupled thereto; and a memory operably coupled to the processor, the memory comprising a library of selectable user interface screens stored therein. The library may include at least one sub-library. The method further includes the step of providing an operator-interface operably connected to the processor and comprising a touch screen. The operator interface receives input commands, displays input command prompts, and transmits at least input commands to the processor. The method also comprises: receiving an input transmission of a selected sub-library; providing at least one input prompt corresponding to the selected sub-library; receiving an input transmission of a selected input command from the touch screen; transmitting the selected input command to the processor; converting the selected input command into an output command; and, transmitting the output command to the pumping apparatus.

### TECHNICAL FIELD

This invention is related to a system and method for operating an infusion pump. More particularly, the invention is directed to an interface and method for operating an infusion pump via a computer program.

### BACKGROUND OF THE INVENTION

It is typical for a medical device to have a display that displays data such as operating parameters. One type of medical device is an infusion pump. Infusion pumps as used in the field of health care are electromechanical devices that control the flow rate of medical fluids. The pumps can deliver drugs at a precise rate, and maintain the drug concentration within a therapeutic margin and out of an unnecessary or possibly toxic range. Generally, the medical pumps provide appropriate drug delivery to the patient at a controllable rate that does not require frequent attention. Types of infusion pumps include syringe pumps, volumetric pumps, and ambulatory pumps.

A syringe pump operates a medical syringe to deliver a drug to a patient at a controlled rate. This pump employs an active fluid pumping mechanism, i.e., positive displacement of the syringe plunger, to expel fluid from the syringe.

A volumetric pump is used with an intravenous fluid administration set having a fluid source and an intravenous (IV) line connecting the fluid source to the patient. The pump is operatively associated with the intravenous line to influence the rate of fluid flow to the patient. For instance, the pump may have a plurality of actuators or fingers for massaging the intravenous line, thus controlling the flow rate of fluid through the line.

Ambulatory pumps typically include a pump control unit and drive mechanism including a variety of operating controllers adapted to accept a disposable pump chamber assembly. The pumping mechanism can include, for example, inlet and outlet valves and a liquid displacement plunger. Each pumping cycle in this type of pump begins with the outlet valve closed and the inlet valve open. Fluid flows from a source container into the section of tubing disposed between the inlet and outlet valve. After this section of tubing has filled with liquid, the inlet valve closes and the outlet valve opens. The plunger then compresses the short section of tubing between the valves, displacing the liquid contained therein and forcing it though the pump.

Pumps such as these often have multiple control panels associated with them for performing various tasks related to both the preparation of the pump, and the administration of medical fluids to a patient. For example, a pump may have one controller for preparing a syringe, a second controller for calibrating the pump, and yet another controller for proceeding with an infusion. The multiplicity of controllers attached to a single pump can be both cumbersome and spatially inefficient. Moreover, different controllers often require separate user training in order to efficiently and effectively operate a pump. This results in increased training time and, ultimately, increased costs to both the service provider and the ultimate recipient of the health services.

The present invention is directed to solving these and other problems.

### SUMMARY OF THE INVENTION

The present invention discloses a system and method for operating an infusion pump. According to an aspect of one embodiment of the present invention, a method for operating an infusion pump comprises the steps of providing a pumping apparatus, the pumping apparatus having an infusion pump and a processor operably coupled to the pumping apparatus. The method further comprises the step of providing a memory operably coupled to the processor. The memory comprises a library of selectable user interface screens stored in the memory. The library may include at least one of a power-up sub-library, a syringe selection sub-library, a pre-programming sub-library, an infusion sub-library, a history sub-library, a device options sub-library, a service sub-library and an advanced set-up sub-library. The method further includes the step of providing an operator interface operably connected to the processor and comprising a touch screen. The operator interface receives input commands, displays input command prompts, and transmits input commands to the processor. The method also comprises the steps of: receiving an input transmission of a selected sub-library; providing at least one input prompt corresponding to the selected sub-library; receiving an input transmission of a selected input command from the touch screen; transmitting the selected input command to the processor; converting the selected input command into an output command; and, transmitting the output command to the pumping apparatus.

According to another embodiment of the present invention, a computer program is disclosed. The computer program operates an infusion pump having a processor adapted to receive input commands from an input device operably coupled thereto, convert the input commands into output commands, and transmit output commands to at least one of a display and a pumping apparatus. The infusion pump operated by the computer program also includes a memory adapted to store a plurality of sub-libraries viewable through user interface screens. The computer program comprises a code segment for receiving an input transmission of a selected sub-library. The program also comprises code segments for receiving an input transmission and transmitting the selected input command to the processor. The program also includes code segments for converting selected input commands into output commands and transmitting the output commands to at least one of a display screen and a pumping apparatus.

According to still another aspect of the present invention, an infusion pump is provided. The infusion pump comprises a touch screen for transmitting input commands and a processor operably connected to the touch screen. The infusion pump also includes a memory coupled to the processor. The memory stores a plurality of sub-libraries which are viewable through a plurality of user interface screens, including a power-up interface screen, a syringe selection interface screen, a pre-programming interface screen, an infusion interface screen, a history interface screen, a device options interface screen, and an advanced set-up interface screen. The infusion pump may also incorporate a computer program stored in the memory which is operably coupled to the processor. The computer program comprises a code segment for receiving an input transmission of a selected sub-library. The program also comprises code segments for receiving an input transmission and transmitting the selected input command to the processor. The program also includes a code segment for converting selected input commands into output commands. The program further includes a code segment for transmitting the output command to either a responsive display screen or pumping apparatus, or both output devices.

According to yet another aspect of the present invention, the infusion pump comprises a pumping apparatus coupled to a syringe, wherein the pumping apparatus is responsive to at least one output command. According to this aspect, the operator interface comprises a display for displaying one of the plurality of software interface screens and output commands transmitted by the processor. The operator interface also includes a touch screen. The touch screen comprises a sensor for sensing a part of the touch screen selected by the user. The sensor also transmits the input command corresponding to the part of the touch screen selected by the user to the processor such that when an input command is received by the touch screen, the input command is transmitted to the processor and the processor converts the input command into an output command.

According to still another aspect of the present invention, an interface for operating an infusion pump is provided. The interface comprises a computer readable program code for prompting and receiving an input transmission of a security access command. In response to the security access command, the code prompts the transmission of a syringe selection input command. In response to the syringe selection input command, the code prompts the transmission of an infusion pump priming input command. The interface also includes a computer readable program code for receiving an infusion pump priming input command, transmitting the infusion pump priming input command to the processor, and converting the infusion pump priming input command into an infusion pump priming output command and transmitting the infusion pump priming output command to the pumping apparatus. The interface further includes a computer readable program code for prompting and receiving the transmission of an infusion pump pre-programming input command. The interface also includes a computer readable program code that transmits the infusion pump pre-programming input command to the processor, and converting the infusion pump pre-programming input command into a pre-programming output command. In response to the pre-programming output command, the code prompts the transmission of an infusion input command. The interface also includes a computer readable program code for receiving an infusion input command, transmitting the infusion input command to the processor, converting the infusion input command into an infusion output command, and transmitting the infusion output command to the pumping apparatus.

These and other advantages will be made apparent from the following description of the drawings and detailed description of the invention.

### BRIEF DESCRIPTION OF THE DRAWINGS

In the drawings, where like numerals refer to like features throughout several views:

FIG. 1 is a front view of a pumping apparatus for use with the present invention;

FIG. 2 is a block diagram of the system of the present invention;

FIG. 3 is a block diagram illustrating a computer of the system in the present invention;

FIG. 4 is a block diagram illustrating one exemplar of a method for using the computer program of FIG. 3;

FIG. 5 is a block diagram illustrating the interface screen library and sub-libraries stored in the memory of the computer of the present invention;

FIGS. 6 and 7 are illustrative examples of various selectable interface screens from the power-up sub-library that may be generated and displayed using the computer program of the present invention;

FIG. 8 is an illustrative example of various selectable interface screens from the syringe selection sub-library that may be generated and displayed using the computer program of the present invention;

FIGS. 9-17 are illustrative examples of various selectable interface screens from the pre-programming sub-library that may be generated and displayed using the computer program of the present invention;

FIGS. 18-20 are illustrative examples of various selectable interface screens from the infusion sub-library that may be generated and displayed using the computer program of the present invention;

FIGS. 21-23 are illustrative examples of various selectable interface screens from the history sub-library that may be generated and displayed using the computer program of the present invention;

FIGS. 24-26 are illustrative examples of various selectable interface screens from the device options sub-library that may be generated and displayed using the computer program of the present invention;

FIGS. 27-30 are illustrative examples of various selectable interface screens from the advanced set-up sub-library that may be generated and displayed using the computer program of the present invention; and,

FIGS. 31A-31F are illustrative examples of various selectable calibration interface screens from the advanced set-up sub-library that may be generated and displayed using the computer program of the present invention.

### DETAILED DESCRIPTION

While this invention is susceptible to embodiments in many different forms, there are shown in the drawings and will herein be described in detail, preferred embodiments of the invention with the understanding that the present disclosures are to be considered as exemplifications of the principles of the invention and are not intended to limit the broad aspects of the invention to the embodiments illustrated.

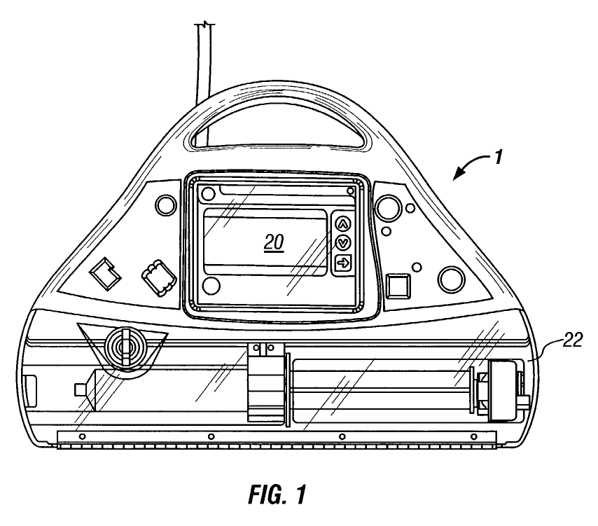

As shown in FIGS. 1-3, the present invention generally includes a computer program for operating an infusion pumping apparatus 22. The system generally comprises a processor 102, an operator interface 20 and a functional pumping apparatus 22. The infusion pump 4 of the pumping apparatus 22 described herein is suitable for controlling the flow rate of medical fluids that are administered to a patient and for delivering drugs at a precise rate to maintain drug concentrations within a therapeutic margin. More particularly, the infusion pump 4 described hereinafter can be embodied in various forms, including, a syringe pump, volumetric pump, or an ambulatory pump. For example, the infusion pump 4 may be of the type described in concurrently filed Application Ser. Nos. ______ (Attorney Docket No. ANP-5786, 1417G P 402) entitled “Infusion Pump,” and ______ (Attorney Docket No. ANP-5693, 1417G P 521) entitled “Dual Orientation Display for a Medical Device.” The entirety of both of these applications are incorporated herein by reference.

If the infusion pump 4 is embodied in a syringe pump, it is known that the pump operates a medical syringe to deliver a drug to a patient at a controlled rate. Accordingly, the syringe pump would employ an active fluid pumping mechanism to expel fluid from the syringe. Alternatively, if the present invention is embodied in a volumetric pump, it is well known in the art that the pump is employed with an intravenous fluid administration set having a fluid source and an intravenous line connecting the fluid source to the patient. The pump according to such an embodiment is operatively associated with the intravenous line to influence the rate of fluid flow to the patient. The present invention may also be embodied in an ambulatory pump. Such pumps include a pump control unit and drive mechanism. Each pumping cycle in this type of pump begins with the outlet valve closed and the inlet valve open. Fluid flows from a source container into the section of tubing disposed between the inlet and outlet valve. After this section of tubing has filled with liquid, the inlet valve closes and the outlet valve opens. The plunger then compresses the short section of tubing between the valves, displacing the liquid contained therein and forcing it though the pump.

Process descriptions or blocks in figures should be understood as representing modules, segments, or portions of code which include one or more executable instructions for implementing specific logical functions or steps in the process, and alternate implementations are included within the scope of the embodiments of the present invention in which functions may be executed out of order from that shown or discussed, including substantially concurrently or in reverse order, depending on the functionality involved, as would be understood by those having ordinary skill in the art.

The operator interface 20 is operably connected to a processor as described in further detail herein below. In one embodiment of the present invention, the operator interface 20 comprises a touch screen 6. The touch screen 6 is generally a graphical user interface (GUI) image having various input mechanisms typically in the form of functional icons. The GUI image also includes a sensor. The sensor senses when a part of the touch screen 6 is selected by a user. Additionally, the sensor transmits an input command corresponding to the part of the touch screen 6 selected by the user to the processor. The operator interface 20 also may also include a display 10. Generally, user interface screens 18 are transmitted to the display 10 of the operator interface 20. Output commands transmitted by the processor are also transmitted to the display 10. It is contemplated by the present invention that the display 10 of the operator interface 20 may be overlaid by the touch screen 6 such that the touch screen 6 is adapted to both receive and display data. Alternatively, the display 10 may be a separate device. Thus, the present invention includes a code segment for generating the GUI touch screen 6 image on the operator interface 20.

The processor 102 also includes a memory 104 that stores a plurality of selectable interface screens 18 that are viewable through the display 10. The plurality of interface screens 18 stored in the memory 104 define an interface screen library 30 comprising a plurality of sub-libraries 32. According to one embodiment of the present invention, the interface screens 18 may include at least one of the following: a power up screen, a security screen, a syringe selection screen, a priming screen, a pre-programming screen, an infusion screen, a history screen, a device options screen, an advanced set-up screen and a service screen. While some of these screens are described in more detail below as comprising specific embodiments of the present invention, it is contemplated that the memory 104 comprises less than all of these screens. It is also contemplated that the invention screen sub-libraries 32 include screens other than those specifically set forth in the embodiments described herein.

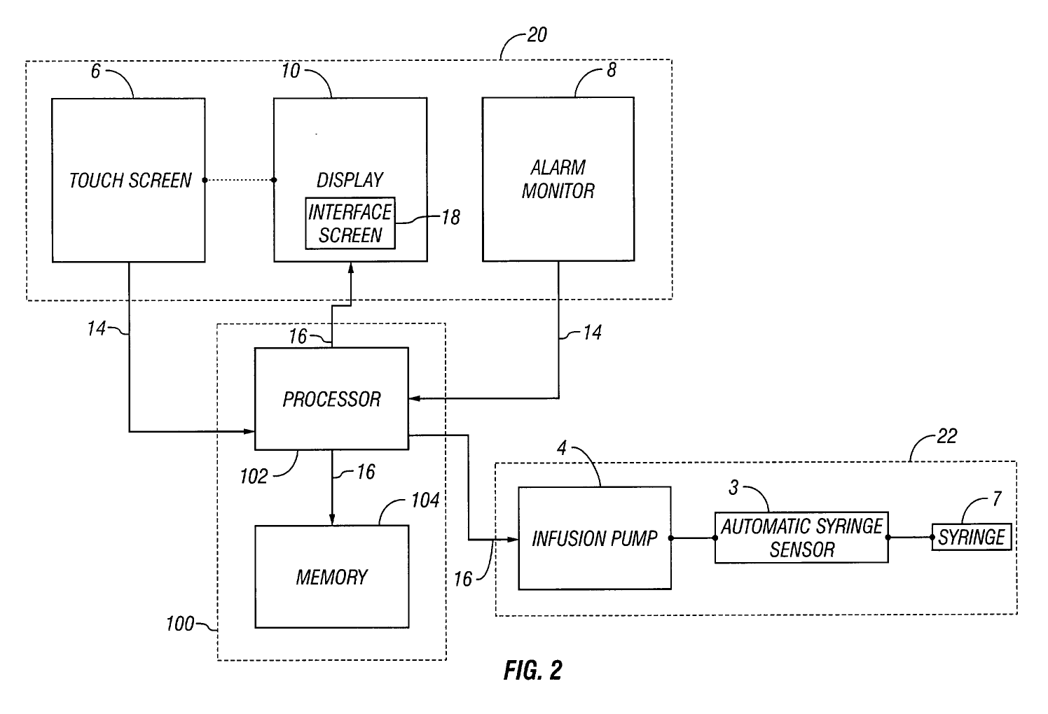

Thus, in the operation of one embodiment of the present invention, and as illustrated in the flowchart of FIG. 2, the processor 102 receives an input command 14 from the operator interface 20 (e.g., a touch screen 6 or an alarm monitor 8). The input command 14 is then translated by the processor 102 into an output command 16. The output command 16 is subsequently sent from the processor 102 to an output device (e.g., the pumping apparatus 22, display 10, memory 104, or all of the above). More particularly, the input command 14 sent by the operator interface 20 dictates the location to which the processor 102 should transmit the corresponding output command 16.

In one embodiment of the present invention shown in FIG. 2, the output message 16 sent to the display 10 from the processor 102 causes a user interface screen 18 to appear on the display 10. The interface screen 18 contains at least one functional icon. Once the interface screen 18 appears on the display 10, the screen can be manipulated by depressing the functional icon through the touch screen 6. Each functional icon corresponds to an input command 14 that is then sent to the processor 102. The input command 14 is then translated by the processor 102 into an output command 16. The output command 16 is then sent from the processor 102 to the pumping apparatus 22, the display 10, the memory 104, or any combination of the preceding objects.

When the output command 16 is sent to the pumping apparatus 22 by the processor 102, the pumping apparatus 22 performs an action 5 responsive to the output command 16. For example, the pumping apparatus 22 may change the rate of an infusion, stop an infusion, start an infusion, or prime the pumping apparatus 22. When the output command 16 is sent to the display 10, a new interface screen 18 from the interface screen library 30 appears. Alternatively, new information appears on the current interface screen 18. When the output message 16 is sent to the memory 104, the output command 16 is stored in the memory 104 until the output command 16 is again accessed by the processor 102. When a stored output command 16 is accessed by the processor 102, the processor 102 sends a copy of the stored output command 16 to the pumping apparatus 22 to perform an action 5 responsive to the output command 16. Alternatively, the processor 102 sends a copy of the stored output command 16 to the display 10 where a screen 18 corresponding to the output command 16 appears.

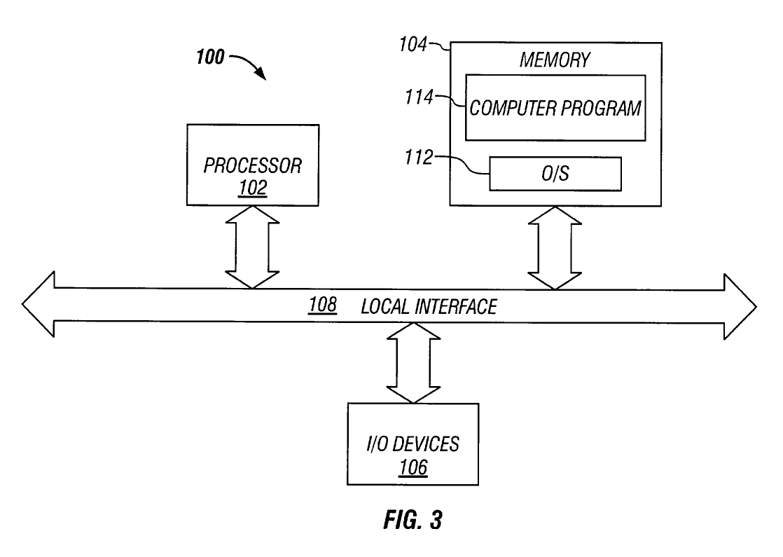

Now is described in more specific terms, the computer hardware associated with operating the computer program 114 of the present invention. FIG. 3 is a block diagram of a computer 100. The computer 100 includes a computer program 114 and operating system 112. The computer program 114 and operating system 112 may reside in, or have portions residing in, any computer such as, but not limited to, a general purpose personal computer. Therefore, computer 100 of FIG. 3 may be appropriately representative of such a computer.

Generally, in terms of hardware architecture, the computer 100 includes a processor 102, memory 104, and one or more input and/or output (I/O) devices 106 that are communicatively coupled via a local interface 108. The local interface 108 can be, for example, but not limited to, one or more buses or other wired or wireless connections, as is known in the art. The local interface 108 may have additional elements, which are omitted for simplicity, such as controllers, buffers (caches), drivers, repeaters, and receivers, to enable communications. Further, the local interface may include address, control, and/or data connections to enable appropriate communications among the other computer components.

The processor 102 is a hardware device for executing software, particularly software stored in memory 104. The processor 102 can be any custom made or commercially available processor, a central processing unit (CPU), an auxiliary processor among several processors associated with the computer 100, a semiconductor based microprocessor (in the form of a microchip or chip set), a macroprocessor, or generally any device for executing software instructions. Examples of suitable commercially available microprocessors are as follows: a PA-RISC series microprocessor from Hewlett-Packard Company, an 80×86 or Pentium series microprocessor from Intel Corporation, a PowerPC microprocessor from IBM, or a SPARC microprocessor from Sun Microsystems, Inc.

The memory 104 can include any one or a combination of volatile memory elements (e.g., random access memory (RAM, such as DRAM, SRAM, SDRAM, etc.)) and nonvolatile memory elements (e.g., ROM, hard drive, tape, CDROM, etc.). Moreover, memory 104 may incorporate electronic, magnetic, optical, and/or other types of storage media. The memory 104 can have a distributed architecture where various components are situated remote from one another, but can be accessed by the processor 102.

The software in memory 104 may include one or more separate programs, each of which comprises an ordered listing of executable instructions for implementing logical functions. In the example of FIG. 3, the software in the memory 104 includes the computer program 114 in accordance with the present invention and a suitable operating system (O/S) 112. The computer program 114 and operating system 112 of the invention can be implemented in software (e.g., firmware), hardware, or a combination thereof. In a preferred embodiment, the computer program 114 and operating system 112 of the present invention are in software, as an executable program, and are executed by one or more special or general purpose digital computer(s), such as a personal computer, personal digital assistant, workstation, minicomputer, or mainframe computer. A non-exhaustive list of examples of suitable commercially available operating systems 112 is as follows: (a) a Windows operating system available from Microsoft Corporation; (b) a Netware operating system available from Novell, Inc.; (c) a Macintosh operating system available from Apple Computer, Inc.; (d) a UNIX operating system, which is available for purchase from many vendors, such as the Hewlett-Packard Company, Sun Microsystems, Inc., and AT&T Corporation; (e) a LINUX operating system, which is freeware that is readily available on the Internet; (f) a run time Vxworks operating system from WindRiver Systems, Inc.; or (g) an appliance-based operating system, such as that implemented in handheld computers or personal digital assistants (PDAs) (e.g., PalmOS available from Palm Computing, Inc., and Windows CE available from Microsoft Corporation). The operating system 112 essentially controls the execution of other computer programs, such as the infusion pump operating system 112, and provides scheduling, input-output control, file and data management, memory management, and communication control and related services.

The infusion pump operating system 112 may be a source program, executable program (object code), script, or any other entity comprising a set of instructions to be performed. When a source program is used, the program may be translated via a compiler, assembler, interpreter, or the like, which may or may not be included within the memory 104, so as to operate properly in connection with the O/S 112. Furthermore, the infusion pump operating system 112 can be written as (a) an object oriented programming language, which has classes of data and methods, or (b) a procedure programming language, which has routines, subroutines, and/or functions, for example but not limited to, C, C++, Pascal, Basic, Fortran, Cobol, Perl, Java, and Ada. In one embodiment, the infusion pump operating system 112 is written in C++. In other embodiments the medical device operating system is created using Power Builder.

The I/O devices 106 may include the touch screen 6, display 10 and alarm monitor 8 of the operator interface 22 devices. The I/O devices 106 may also include other input devices, for example but not limited to, a keyboard, mouse, scanner, microphone, interfaces for various medical devices, bar code readers, stylus, laser readers, or radio-frequency device readers. Furthermore, the I/O devices 106 may also include output devices, for example but not limited to, a printer, bar code printers, or displays. Finally, the I/O devices 106 may further include devices that communicate both inputs and outputs, for instance but not limited to, a modulator/demodulator (modem, for accessing another device, system, or network), a radio frequency (RF) or other transceiver, a telephonic interface, a bridge, a router, etc.

If the computer 100 is a PC, workstation, PDA, or the like, the software in the memory 104 may further include a basic input output system (BIOS) (not shown in FIG. 3). The BIOS is a set of essential software routines that initialize and test hardware at startup, start the O/S 112, and support the transfer of data among the hardware devices. The BIOS is stored in ROM so that the BIOS may be executed when the computer 100 is activated.

When the computer 100 is in operation, the processor 102 is configured to execute software stored within the memory 104, to communicate data to and from the memory 104, and to generally control operations of the computer 100 pursuant to the software. The infusion pump operating system 112 and the O/S 112, in whole or in part, but typically the latter, are read by the processor 102, perhaps buffered within the processor 102, and then executed.

When the infusion pump operating system 112 is implemented in software, as is shown in FIG. 3, it should be noted that the infusion pump operating system 112 can be stored on any computer readable medium for use by or in connection with any computer related system or method. Specifically, the infusion pump operating system 112 can be embodied in any computer-readable medium for use by or in connection with an instruction execution system, apparatus, or device. For example, the system may be embodied in a computer-based system, processor-containing system, or other system that can fetch the instructions from the instruction execution system, apparatus, or device and execute the instructions.

A computer-readable medium can be any means that can store, communicate, propagate, or transport the program for use by or in connection with the instruction execution system, apparatus, or device. The computer readable medium can be for example, but not limited to, an electronic, magnetic, optical, electromagnetic, infrared, or semiconductor system, apparatus, device, or propagation medium. More specific examples (a non-exhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical). Note that the computer-readable medium could even be paper or another suitable medium upon which the program is printed, as the program can be electronically captured, via, for instance, optical scanning of the paper or other medium, then compiled, interpreted or otherwise processed in a suitable manner if necessary, and then stored in memory 104.

In another embodiment, where the infusion pump operating system 112 is implemented in hardware, the infusion pump operating system 112 can be implemented with any or a combination of the following technologies, which are each well known in the art: a discrete logic circuit(s) having logic gates for implementing logic functions upon data signals, an application-specific integrated circuit (ASIC) having appropriate combinational logic gates, a programmable gate array(s) (PGA), a field programmable gate array (FPGA), etc.

Thus, the present invention includes code segments for performing the operation of the device described above. More specifically, the present invention includes a code segment for receiving input commands from the operator interface 20, a code segment for converting the input commands into output commands, and a code segment for transmitting output commands to an output device. Again, the output device may be a pumping apparatus 22, a display 10, a memory 104, or any combination of the preceding. However, it is contemplated that the output device be any other device suited for such an application, including peripheral pump devices.

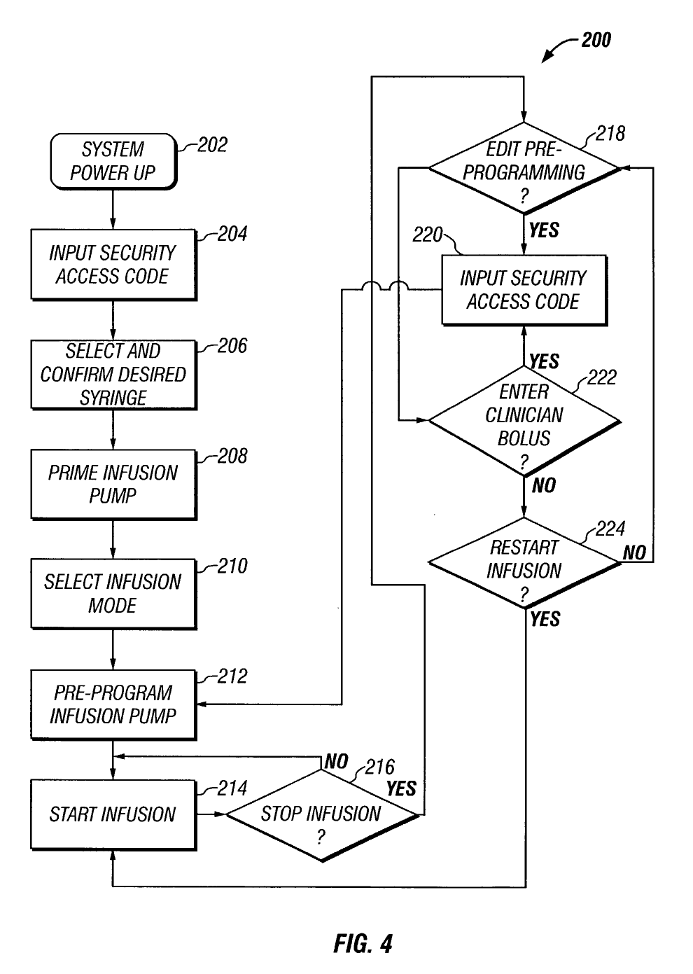

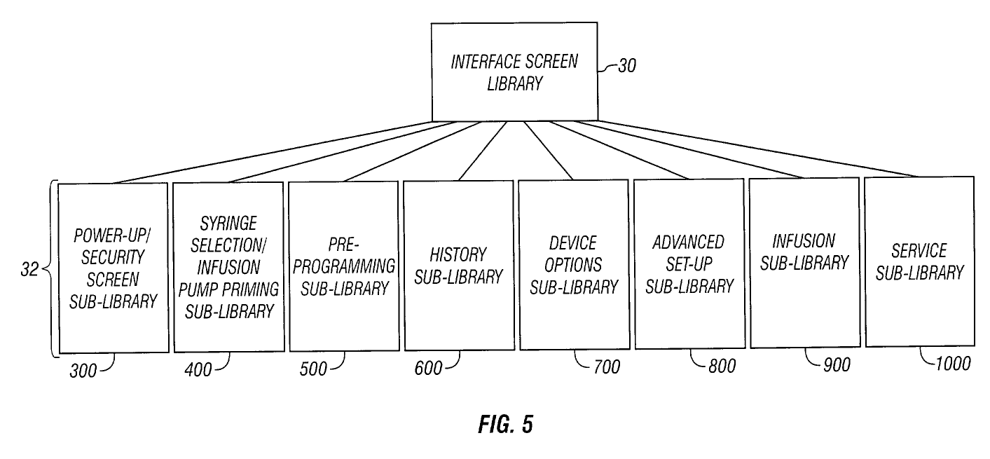

FIG. 4 is a flowchart illustrating one exemplar embodiment of the infusion pump operating system of FIGS. 2 and 3. The computer program 114 of FIG. 3 is called in block 202 by providing power to the system. After the computer program 114 is called in block 202, the system moves to block 204. In block 204, the system accepts input of a security code entered at the operator interface 20. The system then moves to block 206. In block 206, the system accepts input of a desired syringe for performing the infusion. The desired syringe is selected at the operator interface 20, and the system then moves to block 208. In block 208, the system accepts input of a prime infusion pump command that is input via the operator interface 20. After block 208, the system then moves to block 210.

At block 210, the system accepts a transmission of an infusion mode. According to one embodiment the system may accept the transmission of one of a basal+patient controlled analgesia (PCA) infusion, a PCA infusion, or a continuous infusion. As discussed below, the subsequent programming of the system for the specified infusion will be correlative with the selected infusion type. The input of the desired infusion mode to block 210 is communicated via the operator interface 20. The system then moves to block 212.

At block 212, the system accepts input transmission of a plurality of pre-programming commands via the operator interface 20 to program the infusion pumping apparatus 22 for a particular infusion procedure. According to one embodiment of the present invention, the pre-programming commands include: unit selection setting, a dosage concentration setting, a dosage quantity setting, a basal setting, a lockout setting and a PCA limit setting (in either a basal+PCA infusion or a PCA infusion), and an infusion rate setting (in a continuous infusion). The system then moves to block 214 which starts the infusion. Block 214 may accept an input transmission that causes the infusion to be interrupted so that the user may move to block 218 and the pre-programming commands may be adjusted. Alternatively, the infusion may be interrupted to program parameters associated with programming a clinician bolus into an infusion procedure.

Now will be described in detail the interface screen library 30 of one embodiment of the present invention which is composed of the various interface screens 18 that may appear on the display 10. In particular, the screen library 30 is divided into a plurality of sub-libraries 32. In one embodiment of the present invention, the sub-libraries 32 include at least: a power-up/security screen sub-library 300, a syringe selection/priming screen sub-library 400, a pre-programming screen sub-library 500, a history screen sub-library 600, a device options screen sub-library 700, an advanced set-up screen sub-library 800, an infusion screen sub-library 900 and a service screen sub-library 1000. Each of the sub-libraries 30 is made up of at least one interface screen 18 that will appear on the display 10.

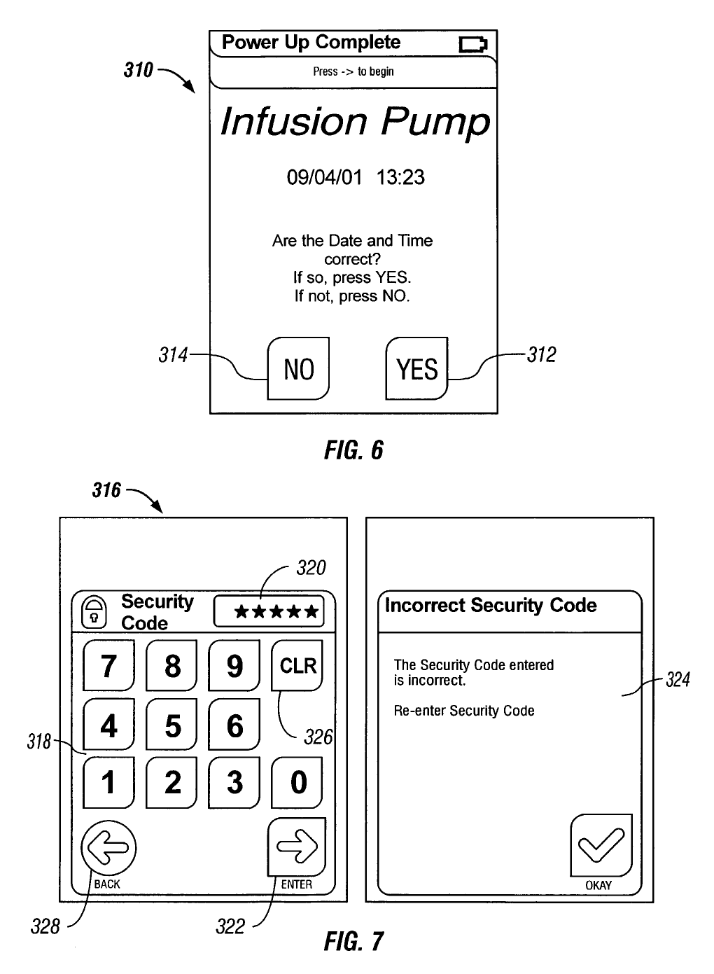

When power is provided to the infusion pump and the pump is turned on, a power-up/security screen sub-library 300 is accessed, and the power-up screen 310 appears on the display 10 (FIG. 6). The power-up screen 310 provides at least two functional icons which query whether the date and time on the display 10 are accurate. The functional icons include at least a “YES” icon 312 and a “NO” icon 314. Depressing either of the icons 312, 314 causes a security screen 316 to appear on the display 10.

In one embodiment of the present invention shown in FIG. 7, the security screen 316 contains a plurality of functional icons displayed as a graphical alpha-numeric keypad 318. The security screen 316 also includes a data bubble 320 for displaying entries input by a user. A user may key in a predetermined security code 24 by depressing alpha-numeric buttons of a graphical keypad 318. The security code must be entered by depressing the correct combination of the functional icons on the keypad 318 and then depressing the “ENTER” icon 322 in order for a new interface screen to appear on the display 10. If the functional icons depressed on the security screen 316 matches the security code, and the “ENTER” icon 322 is depressed, a new command screen will appear on the display 10. If the security code is not entered correctly, the security screen 316 will remain on the display 10. Alternatively, a new interface screen such as shown in FIG. 7 will appear which includes a text message 324 indicating that access has been denied. In the embodiment shown in FIG. 7, a “CLEAR” icon 326 and a “BACK” icon 328 are also provided. If the “CLEAR” icon 326 is depressed, the data input keyed in by the user will be cleared and the data bubble 320 will be reset to appear blank. Alternatively, if the “BACK” icon 328 is depressed, the power-up screen 310 reappears on the display 10. The predetermined security code is stored in the memory 104 of the system. If the security screen 316 was entered by selecting the “YES” icon 312 on the previous screen, the syringe/selection priming screen sub-library 400 will be entered. If, however, the “NO” icon 312 was depressed on the previous screen, the user moves to a Set Date screen.

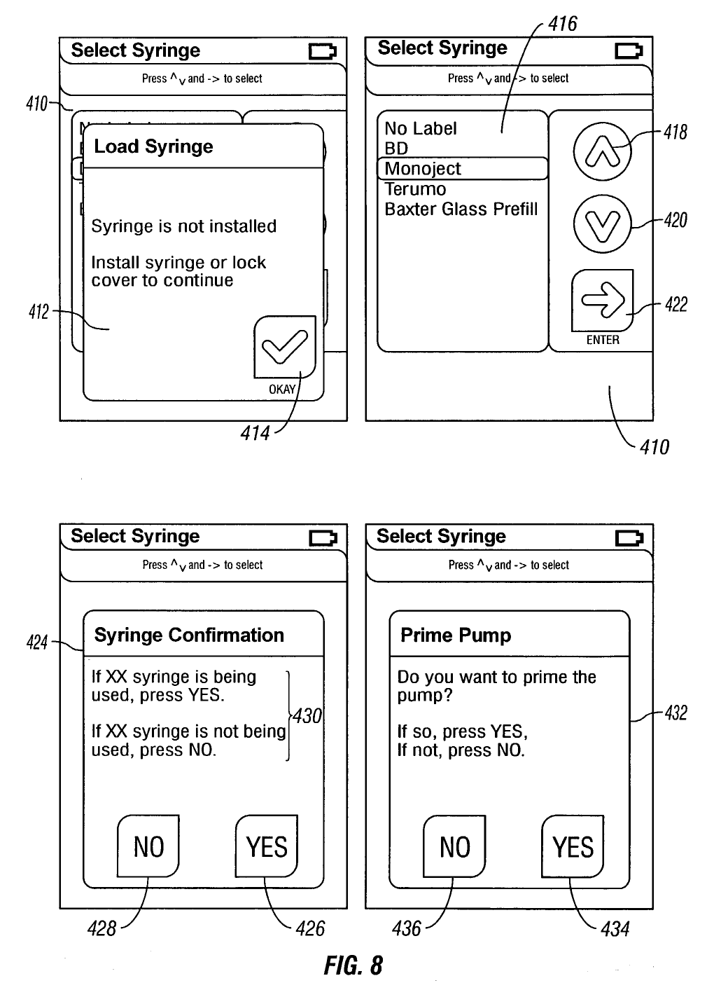

The syringe/selection priming screen sub-library 400 comprises a plurality of command screens relating to selecting a syringe for an infusion procedure and priming the syringe before beginning infusion. As shown in FIG. 2, the pumping apparatus 22 includes an auto-sensor 3 that detects the presence or absence of a syringe 7. If the auto-sensor 3 identifies the syringe 7 connected to the infusion pump 4, the syringe confirmation screen 140 appears on the display 10. If the auto-sensor 3 does not identify the syringe 7 connected to the infusion pump 4, the select syringe screen 410, an example of which is shown in FIG. 8, appears on the display 10 with a text message overlay 412 and a functional “OKAY” icon 414. Depressing the “OKAY” icon 414 causes the text message 412 to disappear. From the select syringe screen 410, the user may select a syringe from a menu 416 containing a plurality of predetermined syringe types. The user may scroll through the menu by depressing either the “TOGGLE-UP” icon 418 or the “TOGGLE-DOWN” icon 420. Once the desired syringe is highlighted, the user presses the “ENTER” icon 422 to select the highlighted syringe, and the syringe confirmation screen 424 appears. If, however, no syringe is loaded, the text message 412 will reappear on the display 10.

Once the auto-sensor 3 identifies a syringe connected to the infusion pump 4, or the “ENTER” icon 422 has been depressed, the syringe confirmation screen 424 appears on the display 10. The syringe confirmation screen 424 contains at least two functional icons: a “YES” icon 426 and a “NO” icon 428. The syringe confirmation screen 424 also displays a text message 430 presenting a query of whether the syringe identified by the auto-sensor 3 is the syringe that is actually attached to the infusion pump 4. From the syringe confirmation screen 424, a user may confirm the selected syringe if the size and brand of the selected syringe is correct by depressing the “YES” icon 426. Depressing the “YES” icon 426 causes the prime pump screen 432 to appear on the display 10. Alternatively, the user may select the “NO” icon 428 if the size and brand of the selected syringe is incorrect. By selecting the “NO” icon 428, the syringe menu 416 will reappear on the display 10. Once in the prime pump screen 432, if the user desires to prime the pump, the user depresses the “YES” icon 434. The pump-priming operation may be aborted by pressing a “STOP” icon in a subsequent screen overlay. If the user does not desire to prime the pump at this time, the user depresses the “NO” icon 426, and the syringe confirmation screen 424 will reappear on the display 10.

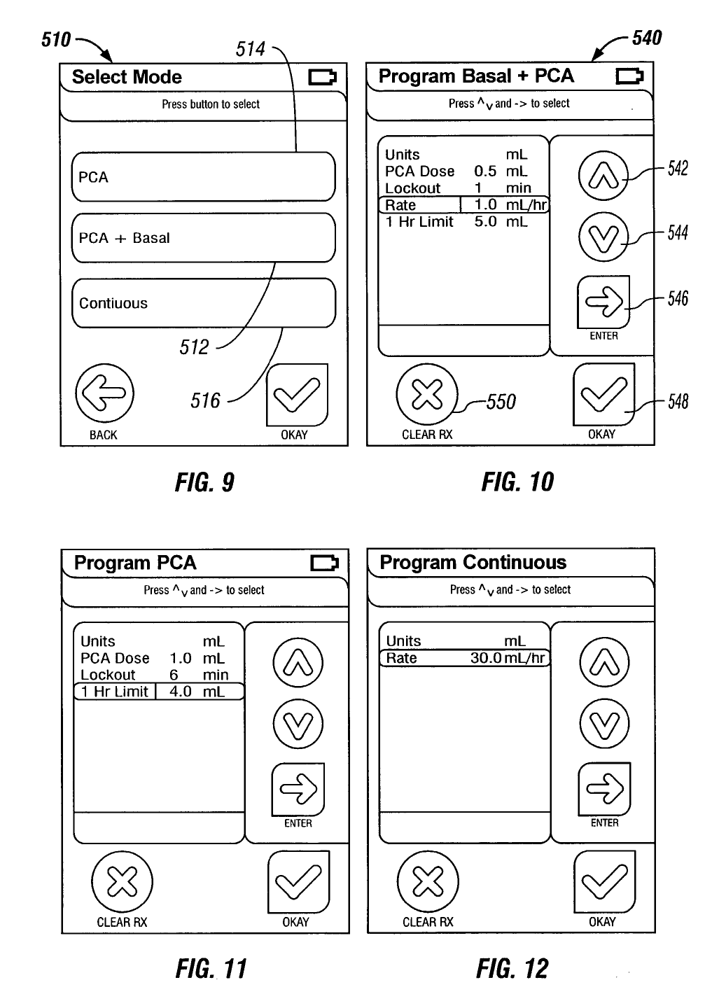

Once the priming operation has been completed, the select mode screen 510 appears and the user enters the pre-programming sub-library 500. As shown for example in FIG. 9, the select mode screen 510 allows a user to select the type of infusion that will occur. Specifically, the user may select a functional icon corresponding to a basal+PCA infusion, a PCA infusion or a continuous infusion as desired. Once one of the “BASAL+PCA INFUSION,” “PCA INFUSION” or “CONTINUOUS INFUSION” icons 512, 514, 516 is selected, the subsequent infusion pre-programming screens corresponding to the selected infusion type appear as described below.

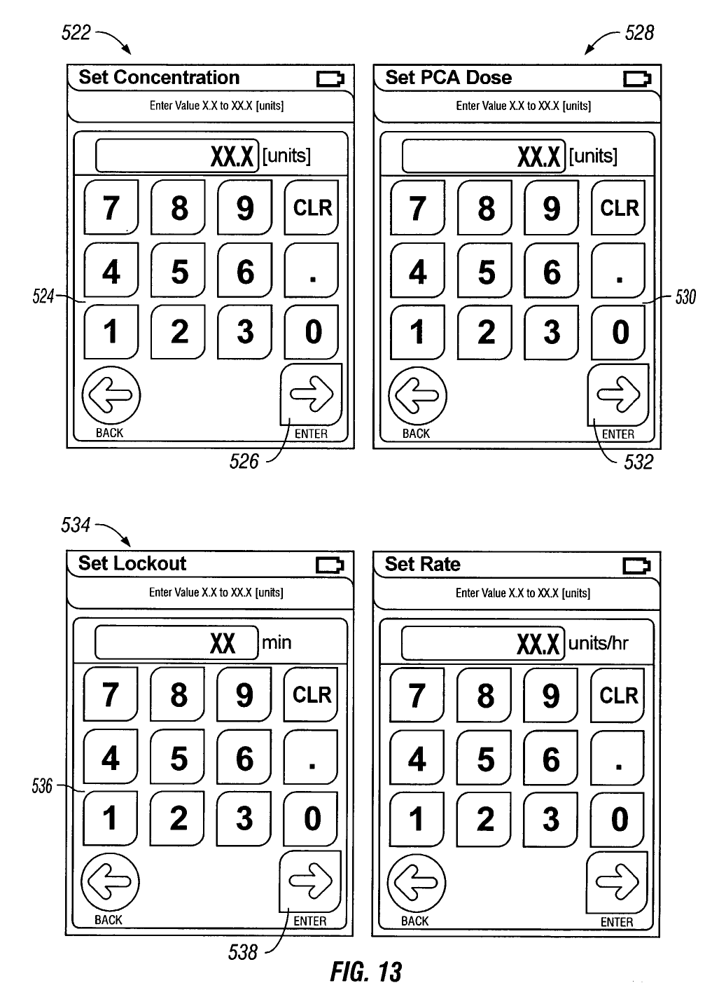

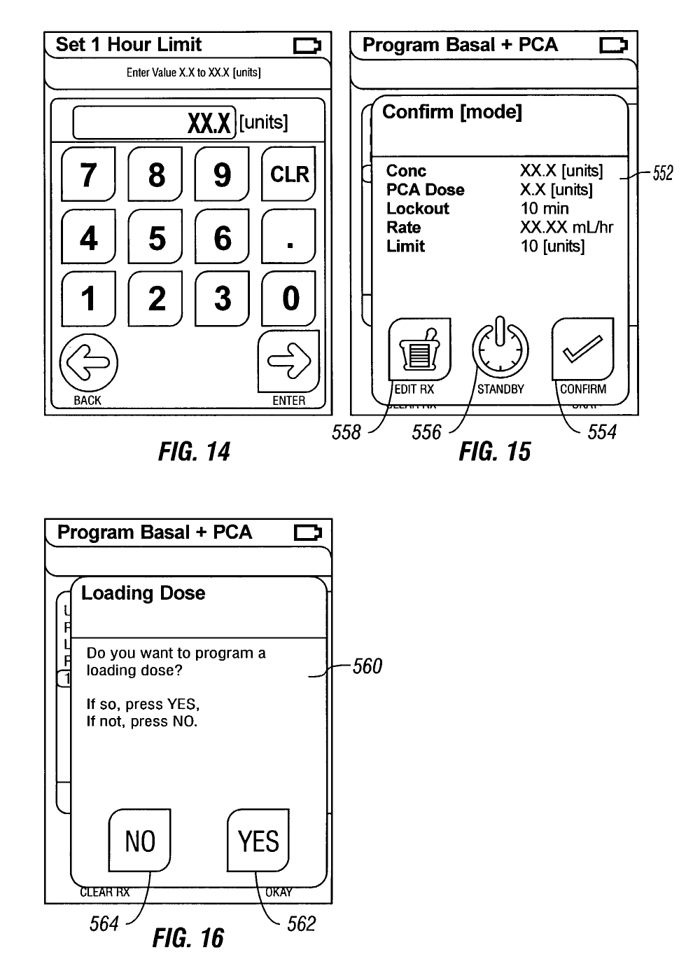

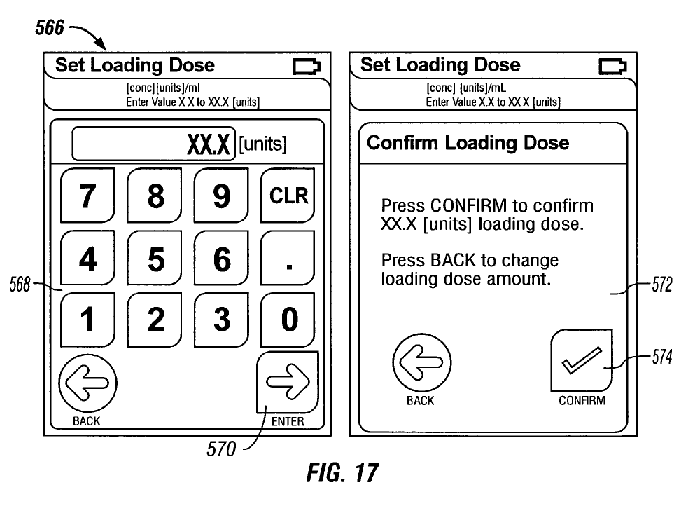

If a user selects the “BASAL+PCA INFUSION” icon, a basal+PCA infusion programming screen 518 appears which allows the user to set the dosage units, dosage volume, concentration, basal and lockout timing within the limits associated with a basal+PCA infusion via the various screens illustrated in FIGS. 9-17. The preferred programmable ranges for a basal+PCA infusion are as follows:

| | |
|---|---|
| PCA dose rate | 150 mL/hr. |
| Loading dose rate | 150 mL/hr. |
| Clinician bolus rate | 150 mL/hr. |
| Basal rate range | 0.0 mL/hr. to 9.9 mL/hr. in 0.1 mL/hr. increments |
| PCA dose volume | 0.1 mL/hr. to 9.9 mL/hr. in 0.1 mL/hr. increments |
| range | |
| Loading dose volume | 0.1 mL/hr. to 9.9 mL/hr. in 0.1 mL/hr. increments |
| range | |
| Clinician bolus dose | 0.1 mL/hr. to 9.9 mL/hr. in 0.1 mL/hr. increments |
| volume range | |
| Delay time range | 3 minutes to 240 minutes |
| Concentration range | 0.1 mg/mL to 99.9 mg/mL in 0.1 mg/mL |
| | increments; |
| | 1 MCG/mL to 9,999 MCG/mL in 1 MCG/mL |
| | increments |

Specifically, a user may select the desired units in which the dosage will be infused (e.g., milliliters, milligrams or micrograms). In the embodiment shown, the units may be selected by highlighting the dosage units. It is, however, contemplated by the present invention that the unit options may be displayed as a drop-down menu or input dialog box. If either milligrams or micrograms are selected, the set concentration screen 522 appears displaying a graphic representation of a numeric keypad 524. The user keys in the desired concentration and then depresses the “ENTER” icon 526 which causes the set dose screen 528 to appear. The set dose screen 528 also includes the graphical numeric keypad 530 which allows a user to key in an appropriate or desired dosage. Once the “ENTER” icon 532 is selected on the set dose screen 528, the set lockout screen 534 appears on the display 10. Again, the user keys in the desired value on a graphical numeric keypad 536, and depresses the “ENTER” icon 538, thereby causing the set basal screen to appear. The user then keys in the desired value on a graphical numeric keypad, and depresses the “ENTER” icon. This causes the set PCA limit screen to appear, in which the user again inputs a desired value using a graphical keypad and is depresses the “ENTER” icon causing a program basal+PCA screen 540 to appear on the display 10.

At the program basal+PCA screen 540, a user may review the programmed basal+PCA infusion parameters. If a change to the infusion must be made, the user may depress either the “UP” icon 542 or the “DOWN” icon 544 to scroll to the desired parameter. Once the desired program is highlighted, the user may depress the “ENTER” icon 546 to return to the programming screen corresponding to the highlighted parameter. Once the programmed basal+PCA infusion is as desired, the user depresses the “OKAY” icon 548 and the confirm prescription screen appears on the display 10. Alternatively, the user may clear the settings by depressing the “CLEAR RX” icon 550.

The confirm prescription screen 552 prompts a user to verify the programmed basal+PCA infusion information. The confirm prescription screen 552 contains a plurality of functional icons, including a “CONFIRM” icon 554, a “STANDBY” icon 556 and an “EDIT RX” icon 558. If the user depresses the “STANDBY” icon 556, the programmed infusion is retained in the memory 104 for future use. After the programmed infusion is stored, or if a user does not wish to store the programmed infusion parameters, the user may move to the loading dose screen 560 by depressing the “CONFIRM” icon. If, however, the user desires to change the programmed infusion parameters, the user depresses the “EDIT RX” icon 558, and the program basal+PCA screen 540 reappears on the display 10.

Once in the loading dose screen 560, a user may either opt to program a loading dose by depressing a “YES” icon 562, or depress the “NO” icon 564 and go directly to a start infusion screen 910. If the user desires to program a loading dose, the user may do so by entering the desired loading dose using a graphical numeric keypad 568 appearing on the set loading dose screen 566, and then depressing the “ENTER” icon 570. This will cause the confirm loading dose screen 572 to appear on the display 10, wherein the user may confirm the programmed loading dose by depressing the “CONFIRM” icon 574.

Alternatively, a user may select the “PCA INFUSION” icon 512. If the “PCA INFUSION” icon 512 is depressed, a PCA infusion programming screen appears which allows the user to set the dosage units, dosage volume, concentration basal and lockout timing within the limits associated with a PCA infusion. The preferred programmable ranges for a PCA infusion are as follows:

| | |
|---|---|
| PCA dose rate | 150 mL/hr. |
| Loading dose rate | 150 mL/hr. |
| Clinician bolus rate | 150 mL/hr. |
| PCA dose volume range | 0.1 mL to 9.9 mL in 0.1 mL increments |
| Loading dose volume range | 0.1 mL to 9.9 mL in 0.1 mL increments |
| Clinician bolus dose volume | 0.1 mL to 9.9 mL in 0.1 mL increments |
| range | |
| Delay time range | 3 minutes to 240 minutes |
| Concentration range | 0.1 mg/mL to 99.9 mg/mL in 0.1 mg/mL |
| | increments; |
| | 1 MCG/mL to 9,999 MCG/mL in |
| | 1 MCG/mL increments |

As with the pre-programming for a basal+PCA infusion, a user may select the desired pre-programming parameters through a plurality of pre-programming sub-library 500 interface screens. It is contemplated that the PCA infusion pre-programming screens be substantially similar to those used in pre-programming the present invention for a basal+PCA infusion. However, it will be known to those skilled in the art that the pre-programming screens for a PCA infusion may include more or fewer screens than those described with respect to the pre-programming sub-library 500 for use in a basal+PCA infusion procedure.

If a user instead selects the “CONTINUOUS” icon, a continuous infusion programming screen appears which allows the user to set the dosage units, dosage concentration and infusion rate within the limits associated with a continuous infusion. The preferred programmable ranges for a PCA infusion are as follows:

| | |
|---|---|
| Loading dose rate | 150 mL/hr. |
| Clinician bolus rate | 150 mL/hr. |
| Rate setting range | 0.1 mL to 99.9 mL in 0.1 mL increments |
| Loading dose volume range | 0.1 mL to 9.9 mL in 0.1 mL increments |
| Clinician bolus dose volume | 0.1 mL to 9.9 mL in 0.1 mL increments |
| range | |
| Concentration range | 0.1 mg/mL to 99.9 mg/mL in 0.1 mg/mL |
| | increments; |
| | 1 MCG/mL to 9,999 MCG/mL in |
| | 1 MCG/mL increments |

As with the pre-programming of either of the previously described basal+PCA infusion and PCA infusions, a user may select the desired pre-programming parameters through a plurality of pre-programming sub-library 500 interface screens. It is contemplated that the PCA infusion pre-programming screens be substantially similar to those used in pre-programming the present invention for a basal+PCA infusion. However, it will be known to those skilled in the art that the pre-programming screens for a PCA infusion may include more or fewer screens than those described with respect to the pre-programming sub-library 500 for use in a basal+PCA infusion procedure.

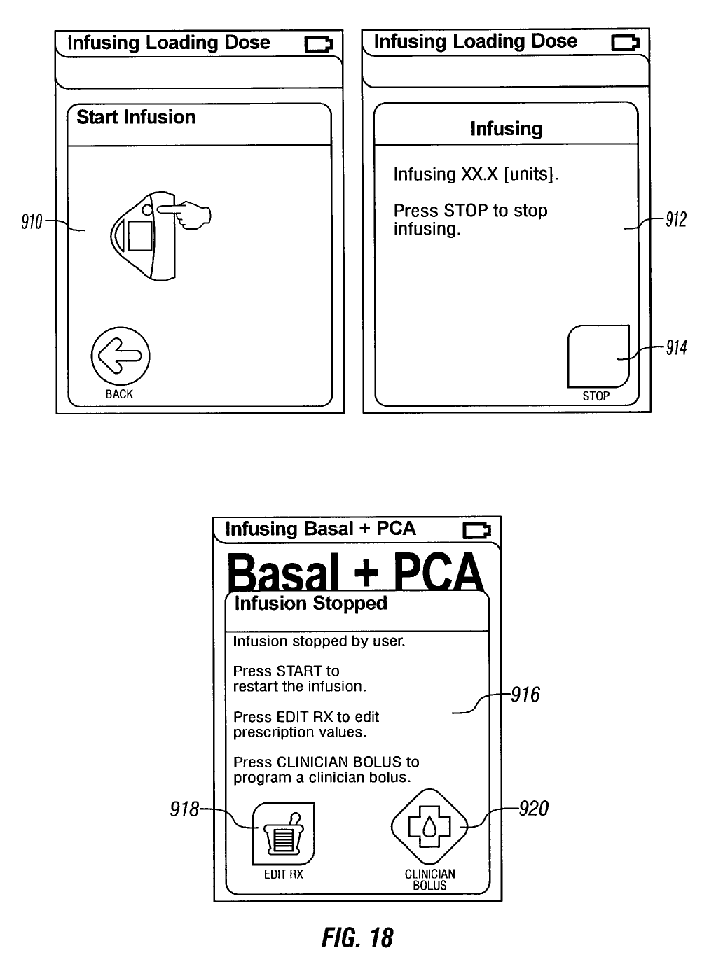

After pre-programming a basal+PCA infusion, PCA infusion, or continuous start infusion via the various interface screens of the pre-programming sub-library 500, the user may be prompted to depress either a functional “START” icon or hardkey to begin the infusion. By depressing the “START” icon or hardkey, the infusion will begin and the system will move to the infusion screen sub-library 900. The infusion screen sub-library 900 contains several interface screens of which FIGS. 18-20 are illustrative.

As shown in FIG. 18, once the infusion begins, an interface screen 912 appears containing a text message which indicates that the desired infusion is in progress. This screen 912 also contains a functional “STOP” icon 914 which allows a user to stop the infusion while in progress. If the infusion is stopped, an infusion stopped screen 916 appears which contains at least two functional icons: an “EDIT RX” icon 918, which returns a user to a security screen 316 to edit programmed infusion parameters; and a “CLINICIAN BOLUS” icon 920, which causes a security screen 316 to appear to program a clinician bolus. The screen 916 may also include a text message that prompts the user to press a “START” hardkey to restart the infusion procedure. Alternatively, the screen 916 may include a functional “START” icon which causes the infusion to restart.

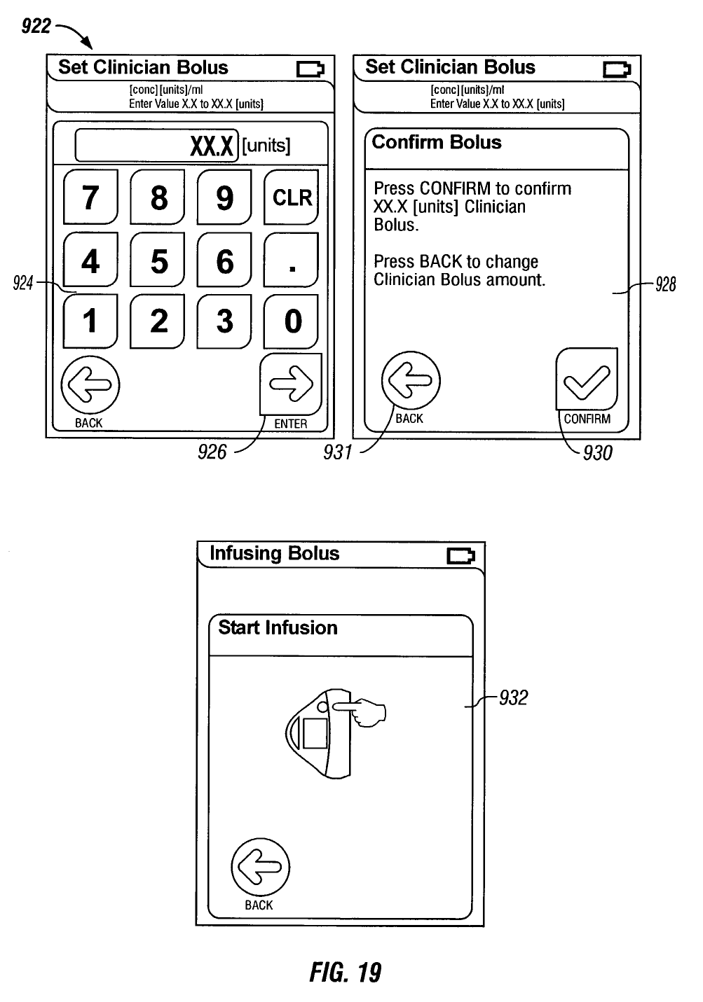

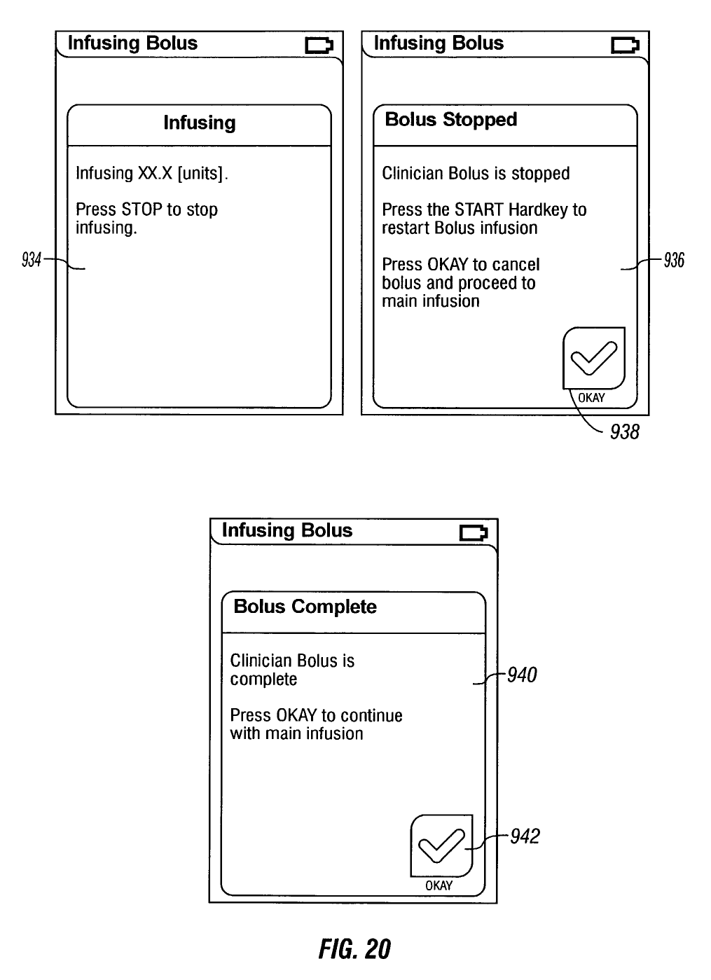

If either the “EDIT RX” or “CLINICIAN BOLUS” icons 918, 920 are depressed, a security screen 316 having an alpha-numeric keypad 318 reappears. As discussed above, the user must enter a predetermined security access code by depressing the appropriate corresponding buttons on the alpha-numeric keypad 318. Once access is confirmed, the appropriate one of the program basal+PCA, program PCA, or program continuous screen reappears (if the “EDIT RX” icon 918 is selected), or a set clinician bolus screen 922 (if the “CLINICIAN BOLUS” icon 920 is selected) will appear. Illustrative examples of interface screens associated with programming a clinician bolus are depicted in FIGS. 19 and 20.

As illustrated in FIG. 19, if the user elects to program a clinician bolus, the clinician bolus screen 922 appears which includes a plurality of functional icons, including a keypad 924 which a user may employ to program a bolus. More particularly, the user enters the desired clinician bolus using the keypad 924 and depresses an “ENTER” icon 926. If the “ENTER” icon 926 is depressed a confirm bolus screen 928 appears. If the user does not wish to proceed with the programmed bolus, the user may depress a “BACK” icon 931, and return to the clinician bolus screen 922. If, however, the user desires to proceed with the programmed bolus, the user may depress an “OKAY” icon 930 on the confirm bolus screen 928, thereby causing a start bolus infusion screen 932 to appear on the display 10. The start infusion screen 932 prompts the user to restart the infusion. If the user elects to proceed with the infusion, a bolus infusing screen 934 appears and the bolus infusion proceeds unless it is stopped by depressing a functional “STOP” icon appearing on the bolus infusing screen 934.

If the bolus infusion is stopped, a bolus stopped screen 936 will appear on the display 10. The bolus stopped screen 936 comprises a text message and at least an “OKAY” icon. Depressing the “OKAY” icon cancels the bolus infusions and causes a main infusion screen 910 to appear on the display 10. Alternatively, the user may depress the “START” hardkey (or functional icon) and restart the bolus infusion. If the user restarts the bolus infusion, or if the bolus infusion is not stopped, the bolus infusion will continue through completion. Once the bolus infusion is complete, a bolus complete screen 940 will appear on the display 10. The bolus complete screen 940 has a functional “OKAY” icon which will move the system to the main infusion screen 910.

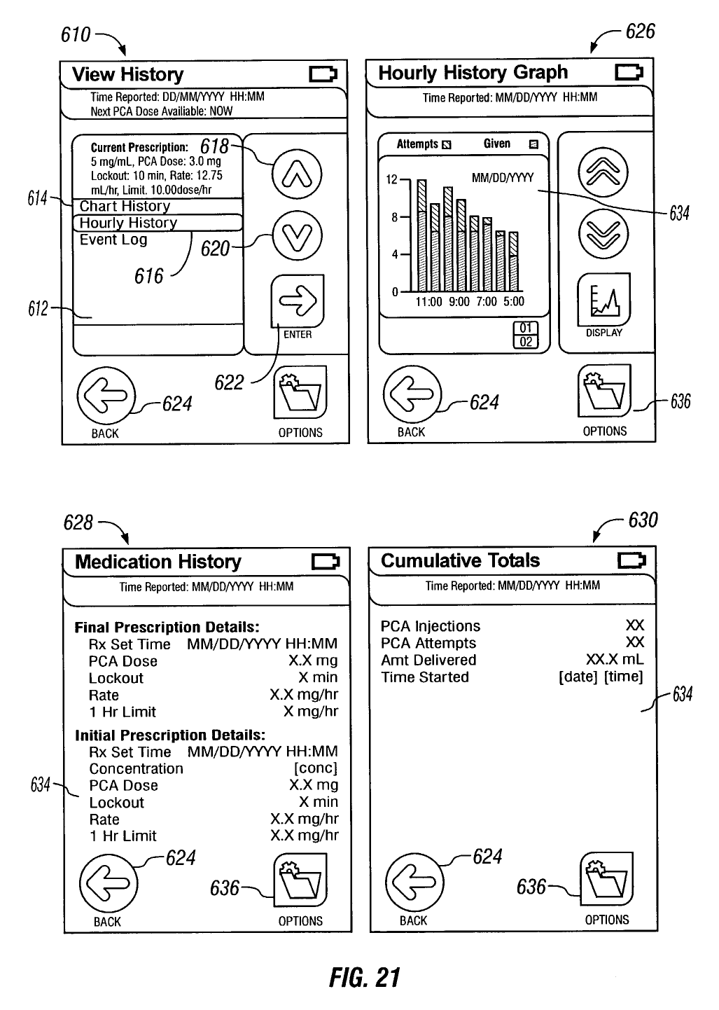

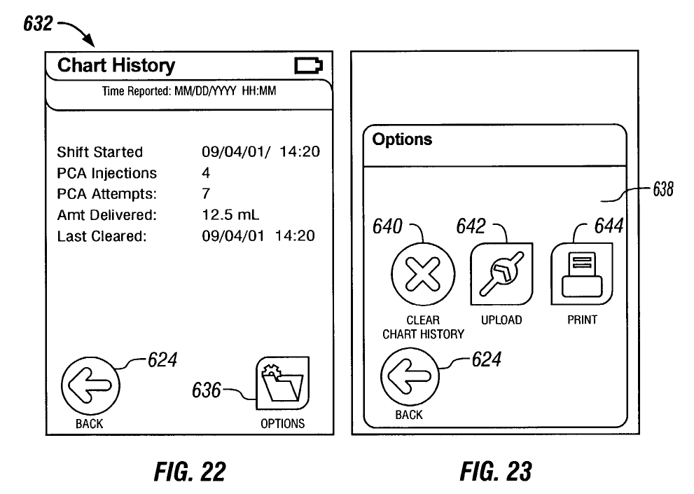

When the history sub-library 600 is selected from the screen library 30, the view history interface screen 610 appears on the display 10. Exemplary embodiments of the interface screens stored in the history sub-library 600 are shown in FIGS. 21-23. As depicted in FIG. 21, one embodiment of the view history screen 610 includes a device history menu 612 comprising screen text 614 and a highlighter 616. The screen text 614 comprises a list of selectable history screen options, including: a chart history, an hourly history, a cumulative history, a final/initial history, an event log and a medication history. Each of the history command screens 626, 628, 630, 632 accessible from the view history screen 610 contains information relevant to the historical operation of the system. It is contemplated that each of the history command screens incorporates selectable text within text window menus. It is further contemplated by the present invention, that each of the history command screens 626, 628, 630, 632 includes a highlighter for selecting desired text within a text window menu.

As shown in FIG. 21, the view history screen 610 further includes a plurality of functional icons, including a “TOGGLE UP” icon 618, a “TOGGLE DOWN” icon 620, an “ENTER” icon 622, and a “BACK” icon 624. In this embodiment, the highlighter 616 may be used to frame one of the history command screen options and can be moved up and down the list of history command screen options by depressing the “TOGGLE UP” and “TOGGLE DOWN” icons 618, 620, respectively.

Depressing the “BACK” icon 624 on any of the screens in the history sub-library 600 will cause the previously displayed screen to reappear on the display 10. When the “ENTER” icon 622 is depressed, the history command screen option currently framed by the highlighter is recognized as the selected history command screen option, and the corresponding history command screen 626, 628, 630, 632 appears on the display 10. Each of the selectable history command screens 626, 628, 630, 632 also includes a text window 634 and a plurality of functional icons. In particular, each of the history command screens includes at least a “BACK” icon 624 (as described above) and an “OPTIONS” icon 636. Depressing the “OPTIONS” icon 636 will cause the history options command screen 638 to appear on the display 10.

The history options display screen 638 provides a plurality of functional icons 640, 642, 644 that will allow a user to clear, upload or print the text displayed on the corresponding history sub-library screen 610, 626, 628, 630, 632. In one embodiment of the present invention, the history options display screen 638 also includes a “BACK” icon 624 which causes the previously displayed history command screen to reappear on the display 10.

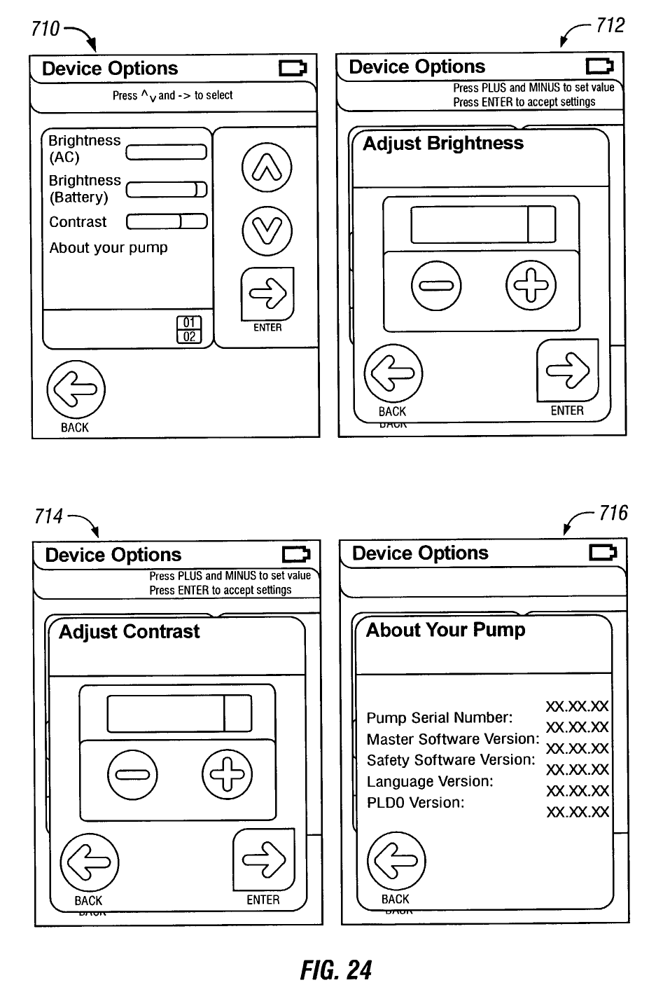

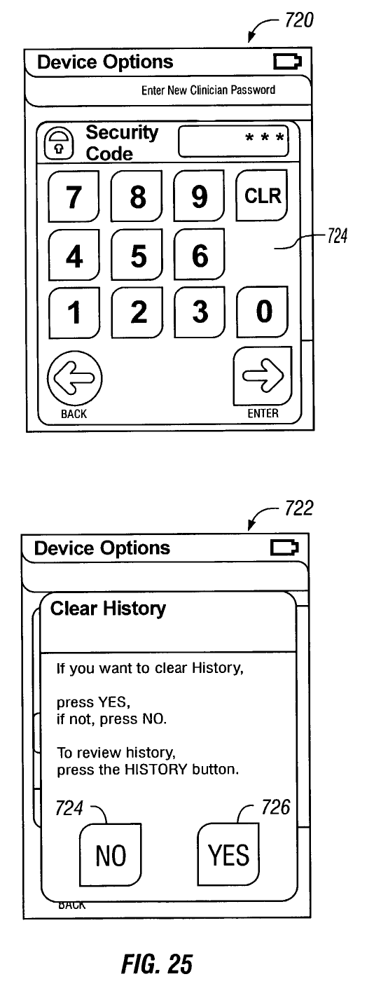

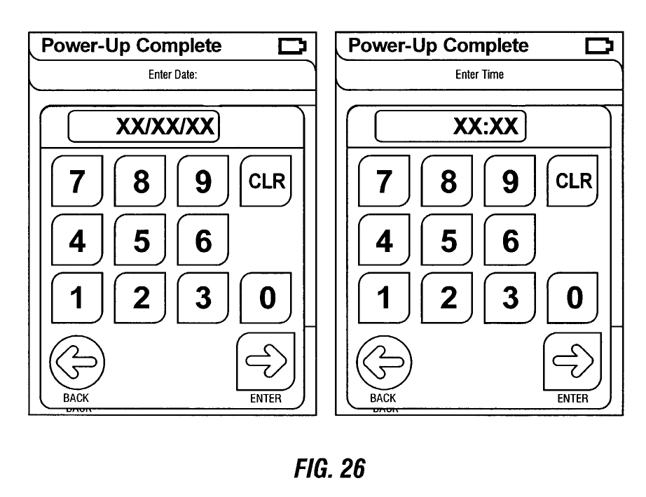

The device options screen sub-library 700, illustrated in FIGS. 24-26, is comprised of a plurality of screens 710, 712, 714, 716, 718, 720, 722 used to adjust various features associated with the display 10. For example, the device options screen sub-library 700 of one embodiment of the present invention includes at least contrast and brightness adjustment screens 712, 714 which allow the user to adjust the contrast and the brightness of the display 10 respectively. The device options screen sub-library 700 may also include a change security password screen 720 comprising an alpha-numeric keypad 724 in which a user may key in a new password to provide security access. The device options screen sub-library 700 may also include screens (e.g., 716) related to obtaining data regarding the pumping apparatus 22. In one embodiment, the device options screen sub-library 700 also includes a clear history screen 722 having a “NO” icon 726 and a “YES” icon 728. Depressing the “NO” icon 726 returns the user to a main device options 710 screen, while depressing the “YES” icon 728 causes the view history screen 610 to reappear, wherein the user is further prompted to confirm that the historical data be deleted.

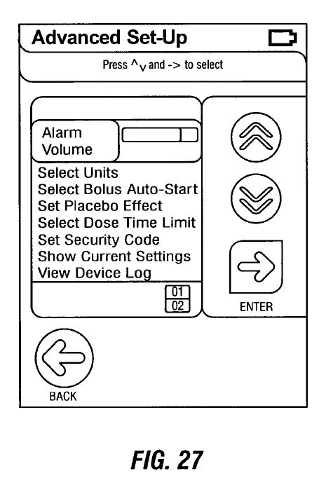

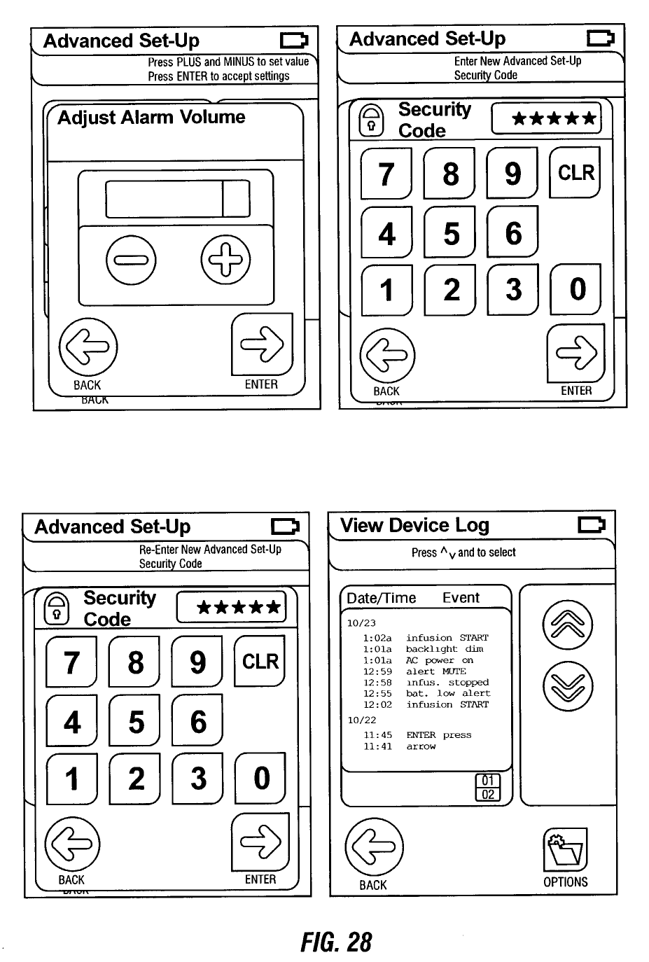

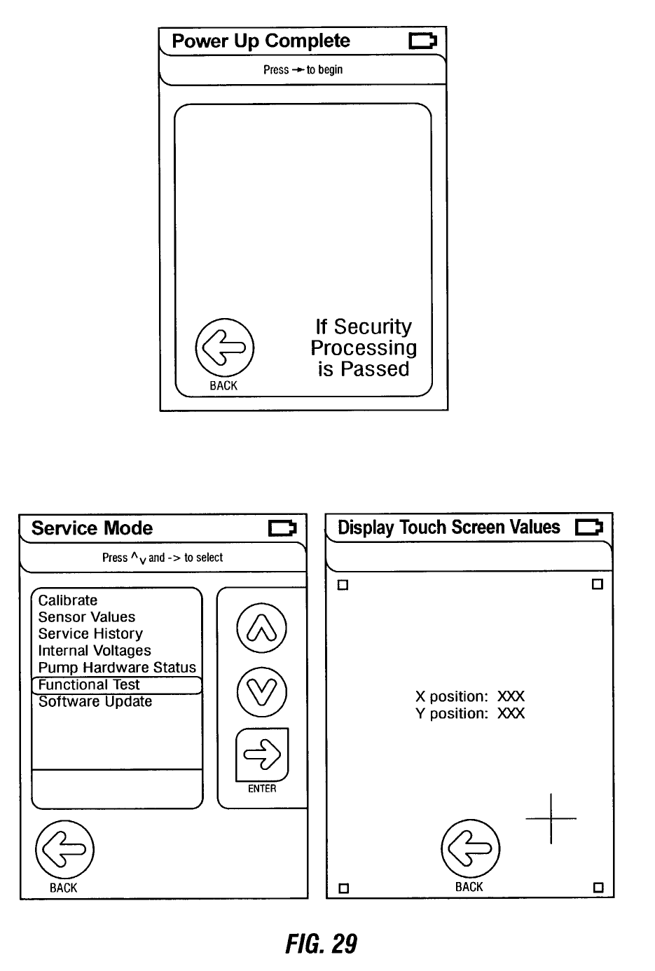

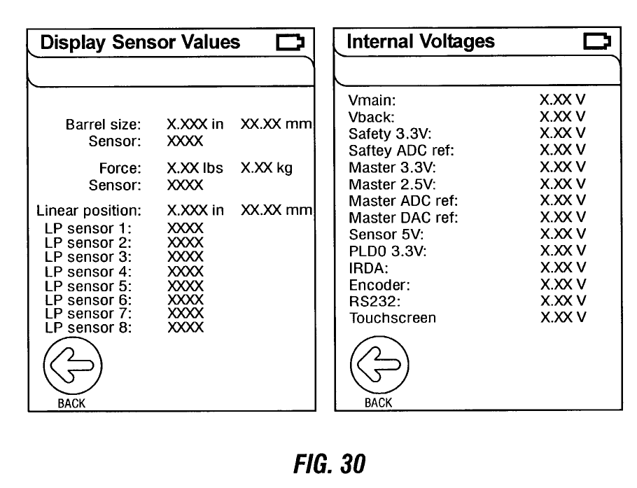

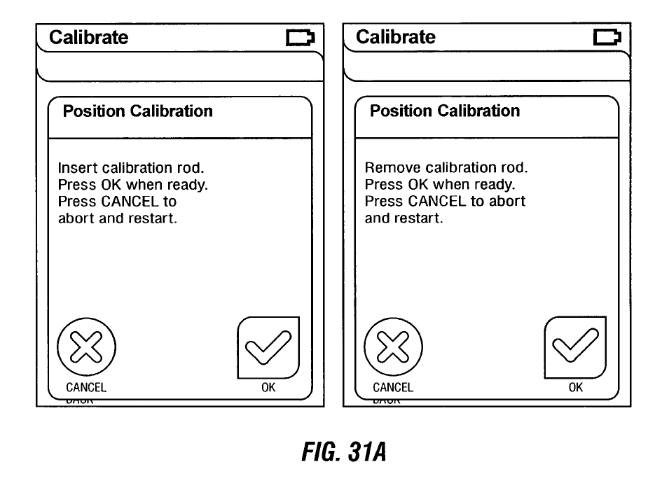

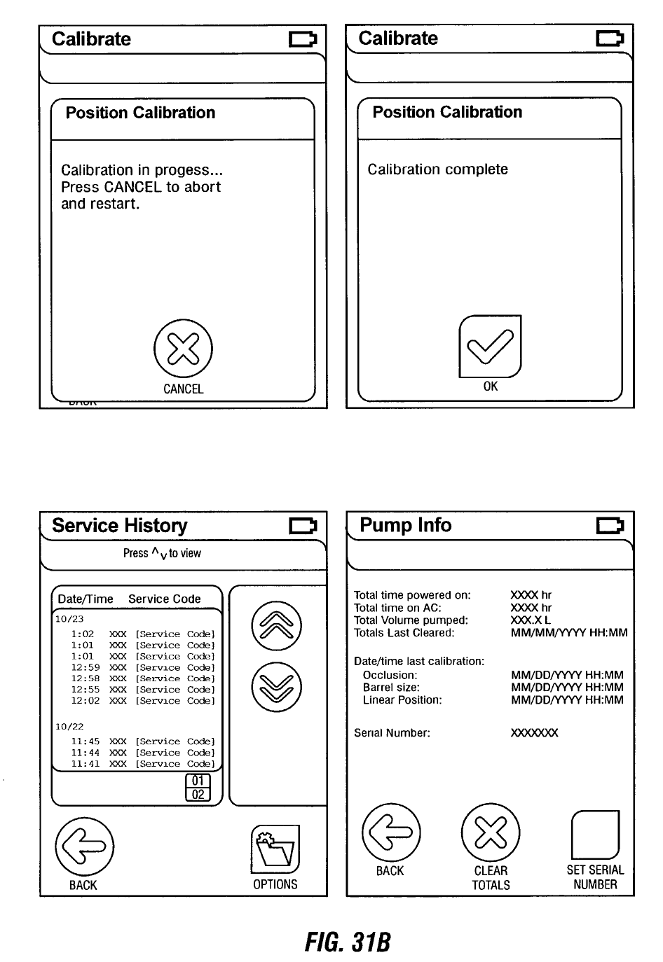

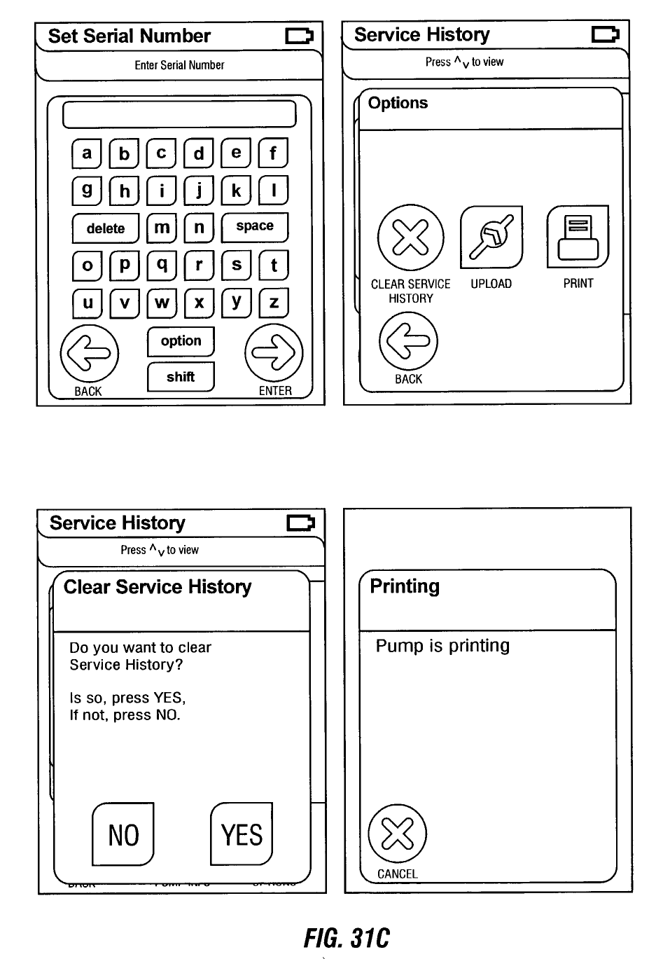

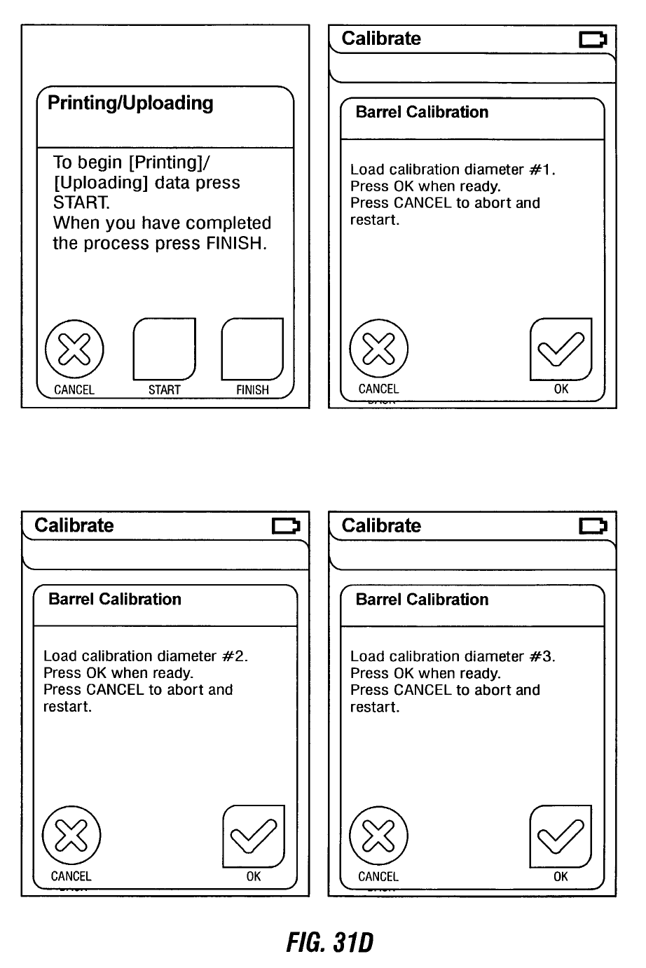

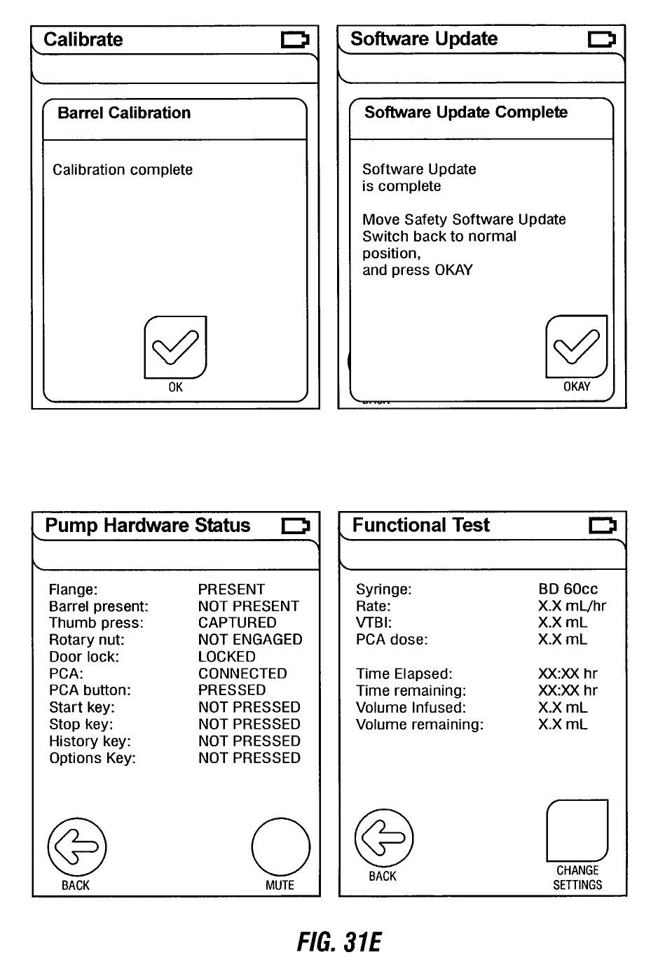

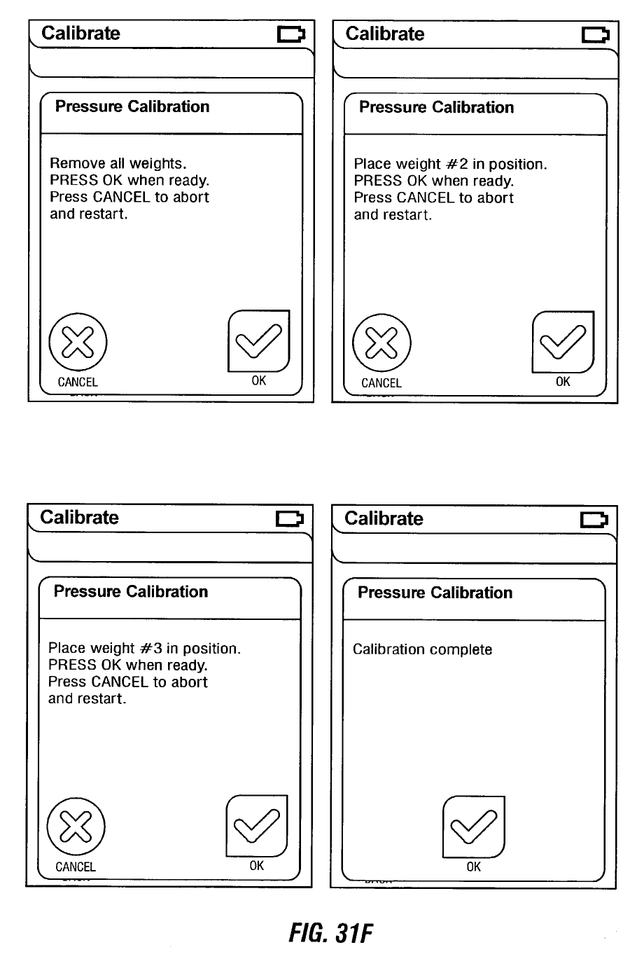

As shown in FIGS. 27-31, the advanced set-up screen sub-library 800 is comprised of a plurality of screens used primarily by a physician or health provider to calibrate the system or to set up certain infusion characteristics. In one embodiment of the present invention, the advanced set-up screen sub-library 800 is comprised of at least: a select units screen, a select bolus auto-start screen, a select placebo effect screen, a select dose limit type screen, a set security code screen, a restore factory default screen, a show current settings screen and a view device log screen. Also, as illustrated in FIGS. 31A-31F, the advance set-up screen sub-library 800 may contain a plurality of screens which allow the user to calibrate the system.

It will be understood that the invention may be embodied in other specific forms without departing from the spirit or central characteristics thereof. The present embodiments, therefore, are to be considered in all respects as illustrative and not restrictive, and the invention is not to be limited to the details given herein.

What is claimed is:

1. A method for operating an infusion pump, the method comprising the steps of:
providing a pumping apparatus, the pumping apparatus having an infusion pump;
providing a processor operably coupled to the pumping apparatus;
providing a memory operably coupled to the processor comprising a library of selectable user interface screens stored in the memory, the library comprising at least one of a power-up sub-library, a syringe selection sub-library, a pre-programming sub-library, an infusion sub-library, a history sub-library, a device options sub-library, a service sub-library and an advanced set-up sub-library;
providing an operator interface for receiving input commands, the operator interface being operably connected to the processor and comprising a touch screen for displaying at least one input command prompt and for transmitting at least one input command to the processor;
receiving an input transmission of a selected sub-library;
providing at least one input prompt corresponding to the selected sub-library;
receiving an input transmission of a selected input command from the touch screen;
transmitting the selected input command to the processor;
converting the selected input command into an output command; and,
transmitting the output command to the pumping apparatus.

2. The method of claim 1, wherein the selected sub-library is the power-up sub-library.

3. The method of claim 2, further comprising the step of prompting an input transmission of a security access command.

4. The method of claim 3, wherein the step of prompting an input transmission of a security access command comprises displaying a selectable keypad image comprising a plurality of functional alphanumeric icons.

5. The method of claim 1, wherein the selected sub-library is the syringe selection sub-library.

6. The method of claim 5, further comprising the step of prompting an input transmission of syringe selection data command.

7. The method of claim 6, wherein the step of prompting an input transmission of a syringe confirmation and selection data command comprises displaying a plurality of functional icons, wherein each functional icons corresponds to a syringe confirmation and selection option.

8. The method of claim 6, further comprising the step of prompting the input transmission input of a syringe priming command.

9. The method of claim 1, wherein the selected sub-library is the pre-programming sub-library.

10. The method of claim 9, further comprising the step of prompting an input transmission of prescription commands responsive to a plurality of drug administering questions.

11. The method of claim 10, wherein the step of prompting the input transmission of prescription commands comprises displaying a plurality of functional icons, wherein each functional icons corresponds to a prescription option responsive to one of the plurality of drug administering questions.

12. The method of claim 10, wherein the step of prompting an input transmission of prescription commands comprises prompting the selection of a prescription medication from a database of a plurality of predetermined prescription medications.

13. The method of claim 1, wherein the selected sub-library is the infusion sub-library.

14. The method of claim 13, further comprising the step of prompting an input transmission of an infusion command responsive to a plurality of drug infusion questions.

15. The method of claim 14, wherein the step of prompting the input transmission of an infusion command comprises displaying a plurality of functional icons, wherein each functional icon corresponds to an infusion option responsive to one of the plurality of drug infusion questions.

16. The method of claim 1, wherein the selected sub-library is the history sub-library, the history sub-library comprising user interface screens corresponding to historical pump data, the historical pump data comprising hourly history data, cumulative history data, final history data, initial history data, medication history data and a chart history data.

17. The method of claim 16, further comprising the step of prompting an input transmission of a history selection command, wherein the history selection command corresponds to one of either hourly history data, cumulative history data, final history data, initial history data, a chart history data, an event log and medication history data.

18. The method of claim 16, further comprising the steps of:
receiving a input transmission of a history selection command; and,
transmitting the historical pump data associated with one of either an hourly history data, cumulative history data, final history data, initial history data, a chart history data and medication history data, and corresponding to the transmitted history selection command, to an output device.

19. The method of claim 1, wherein the selected sub-library is the device option sub-library.

20. The method of claim 19, further comprising the step of prompting an input transmission of one of a plurality of device option commands, the step comprising displaying a plurality of functional icons corresponding to settings relevant to the operator interface.

21. The method of claim 20, further comprising the step of:
receiving an input transmission of one of a plurality of device option commands;
transmitting the device option command to the processor;
converting the input device option command into an output device option command;
transmitting the infusion command to the operator interface; and, executing the device option command.

22. The method of claim 1 further including the step of providing an alarm monitor operably connected to the processor, the alarm monitor being responsive to at least one predetermined condition, wherein the at least one predetermined condition causes the alarm monitor to generate an input command and transmit the input command to the processor.

23. The method of claim 1 further including the step of providing a display for displaying output commands generated by the processor, the display being operably coupled to the processor.

24. The method of claim 23, wherein the touch screen overlays the display.

25. A computer program for operating an infusion pumping apparatus, wherein the infusion pump comprises a processor adapted to receive input commands from an input device operably coupled thereto, to convert the input commands into output commands, and to transmit output commands to at least one of a display and pump, and wherein the infusion pumping apparatus further comprises a memory adapted to store a plurality of sub-libraries containing user interface screens that are viewable through a display, the computer program comprising:
a first code segment for receiving an input transmission of a selected sub-library;
a second code segment for receiving an input transmission of a selected input command from the input device;
a third code segment for transmitting the selected input command to the processor;
a fourth code segment for converting the selected input command into an output command; and,
a fifth code segment for transmitting the output command to at least one of a display and a pumping apparatus.

26. The computer program of claim 25, wherein the input device comprises a touch screen for displaying at least one input command prompt and for transmitting at least one input command.

27. The computer program of claim 25, wherein the input device is an alarm monitor operably connected to the processor, the alarm monitor being responsive to at least one predetermined condition, wherein the at least one predetermined condition causes the alarm monitor to generate an input command which is transmitted to the processor.

28. The computer program of claim 25, wherein the plurality of sub-libraries comprises at least one of a power-up interface screen, a syringe selection interface screen, a pre-programming interface screen, an infusion interface screen, a history interface screen, a device options interface screen, and an advanced set-up interface screen.

29. The computer program of claim 25, further comprising a code segment for prompting an input transmission of a security access command, wherein the code segment generates a graphical user interface image of a selectable keypad screen comprising a plurality of functional icons.

30. The computer program of claim 25, further comprising a code segment for prompting an input transmission of a syringe selection command, wherein the code segment generates a graphical user interface image comprising a plurality of functional icons, wherein each functional icon corresponds to a syringe selection option.

31. The computer program of claim 25, further comprising a code segment for prompting an input transmission of prescription commands, wherein the code segment generates a graphical user interface image comprising a plurality of functional icons, wherein each functional icon corresponds to a prescription option responsive to one of the plurality of drug administering questions.

32. The computer program of claim 31, further comprising a database storing a plurality of predetermined prescription medications.

33. The computer program of claim 25, further comprising a code segment for prompting an input transmission of an infusion command, wherein the code segment generates a graphical user interface image comprising a plurality of functional icons, wherein each functional icon corresponds to an infusion option responsive to one of the plurality of drug infusion questions.

34. The computer program of claim 28, wherein the history sub-library further comprises a memory, the memory comprising historical pump data, wherein the historical pump data comprises at least one of a shift history, an hourly history, an event history, a settings history and a complete history.

35. The computer program of claim 34, further comprising a code segment for prompting an input transmission of a history selection command, wherein the code segment generates a graphical user interface image comprising a plurality of functional icons, wherein each functional icon is operably coupled to a database storing one of a hourly history data, cumulative history data, final history data, initial history data, an event log and medication history data.

36. The computer program of claim 25, further comprising a code segment for prompting an input transmission of one of a plurality of device option commands, wherein the code segment generates a graphical user interface image comprising a plurality of functional icons corresponding to settings relevant to the operator interface.

37. An infusion pump comprising:
a touch screen for transmitting input commands;
a processor operably connected to the touch screen;
a memory coupled to the processor, the memory having a plurality of sub-libraries stored therein, the sub-libraries being viewable through a plurality of user interface screens comprising at least one of a power-up interface screen, a syringe selection interface screen, a pre-programming interface screen, an infusion interface screen, a history interface screen, a device options interface screen, a service sub-library and an advanced set-up interface screen; and,
a computer program stored in the memory and being operably coupled to the processor, the computer program comprising:
a first code segment for receiving an input transmission of a selected sub-library;
a second code segment for receiving an input transmission of a selected input command from the touch screen;
a third code segment for transmitting the selected input command to the processor;
a fourth code segment for converting the selected input command into an output command; and,
a fifth code segment for transmitting the output command to at least one of a display and
a pumping apparatus, the at least one display and pumping apparatus being responsive to the output command.

38. The system of claim 37, further comprising an alarm monitor operably connected to the processor, the alarm monitor being responsive to at least one predetermined condition, wherein the at least one predetermined condition causes the alarm monitor to generate an input command which is transmitted to the processor.

39. The system of claim 37, wherein the computer program further comprises a code segment for prompting an input transmission of a security access command, wherein the code segment generates a graphical user interface image of a selectable keypad screen comprising a plurality of functional icons.

40. The system of claim 37, wherein the computer program further comprises a code segment for prompting an input transmission of syringe confirmation and selection data command, wherein the code segment generates a graphical user interface image comprising a plurality of functional icons, wherein each functional icon corresponds to a syringe confirmation and selection option.

41. The system of claim 37, wherein the computer program further comprises a code segment for prompting an input transmission of prescription commands, wherein the code segment generates a graphical user interface image comprising a plurality of functional icons, wherein each functional icon corresponds to a prescription option responsive to one of the plurality of drug administering questions.

42. The system of claim 37, wherein the computer program further comprises a database storing a plurality of predetermined prescription medications.

43. The system of claim 37, wherein the computer program further comprises a code segment for prompting an input transmission of an infusion command, wherein the code segment generates a graphical user interface image comprising a plurality of functional icons, wherein each functional icon corresponds to an infusion option responsive to one of the plurality of drug infusion questions.

44. The system of claim 37, wherein the history sub-library comprises user interface screens corresponding to historical pump data, the historical pump data comprising hourly history data, cumulative history data, final history data, initial history data, a chart history data and medication history data.

45. The system of claim 44, wherein the computer program further comprises a code segment for prompting an input transmission of a history selection command, wherein the code segment generates a graphical user interface image comprising a plurality of functional icons, wherein each functional icon is operably coupled to a database storing one of a hourly history data, cumulative history data, final history data, initial history data, an event log and medication history data.

46. The system of claim 37, wherein the computer program further comprises a code segment for prompting an input transmission of one of a plurality of device option commands, wherein the code segment generates a graphical user interface image comprising a plurality of functional icons corresponding to settings relevant to the operator interface.

47. An interface for operating an infusion pump, wherein the infusion pump comprises a processor adapted to receive input commands from an operator interface, to convert the input commands into output commands, and to transmit output commands to at least one of a display and a pumping apparatus, the pump comprising a memory operably coupled to the processor, wherein the memory is adapted to store a plurality of sub-libraries viewable through software interface screens, the plurality of sub-libraries comprising at least one of a pre-programming interface screen, a programming interface screen, an infusion group interface screen, a history interface screen, a device options interface screen, an advanced set-up interface screen, and an alert group interface screen, the interface comprising computer readable program code for:
prompting the transmission of a security access command;
receiving an input transmission of a security access command;
in response to the security access command, prompting the transmission of syringe selection input command;
in response to the syringe selection input command, prompting the transmission of an infusion pump priming input command;
receiving an infusion pump priming input command;
transmitting the infusion pump priming input command to the processor;
converting the infusion pump priming input command into an infusion pump priming output command;
transmitting the infusion pump priming output command to the pumping apparatus;
prompting the transmission of an infusion pump pre-programming command;
receiving an infusion pump pre-programming input command;
transmitting the infusion pump pre-programming input command to the processor;
converting the infusion pump pre-programming input command into a pre-programming output command;
in response to the pre-programming output command, prompting the transmission of an infusion input command;
receiving an infusion input command;
transmitting the infusion input command to the processor;
converting the infusion input command into an infusion output command; and,
transmitting the infusion output command to the pumping apparatus.

48. The interface of claim 47, further comprising computer readable program code for:
prompting a transmission of a pre-programming edit input command;
receiving a pre-programming edit input command;
transmitting the pre-programming edit input command to the processor;
converting the pre-programming edit input command into a pre-programming edit output command; and,
transmitting the edit output command to the pumping apparatus.

49. The interface of claim 47, further comprising computer readable program code for prompting a transmission of a clinician bolus programming input command;
transmitting the clinician bolus programming input command to the processor;
converting the clinician bolus programming input command into a clinician bolus programming output command; and,
transmitting the clinician bolus programming output command to the pumping apparatus.

50. An infusion pump comprising:
a processor;
a memory coupled to the processor;
a computer program stored in the memory and accessible by the processor, the computer program comprising a code segment for receiving input commands from an operator interface, a code segment for converting the input commands into output commands, and a code segment for transmitting output commands to at least one of a display and a pumping apparatus, the processor further comprising a memory stored in the processor, the memory comprising a code segment for storing a plurality of software interface screens, the plurality of software interface screens comprising at least one of a power-up interface screen, a security interface screen, a syringe selection interface screen, a pre-programming interface screen, an infusion interface screen, a history interface screen, a device options interface screen, and an advanced set-up interface screen;
a pumping apparatus coupled to a syringe, the pumping apparatus being responsive to at least one output command; and,
an operator interface for receiving and transmitting input commands, the operator interface being operably connected to the processor and comprising:
a display for displaying one of the plurality of software interface screens and output commands transmitted by the processor; and,
a touch screen, the touch screen comprising a sensor for sensing a part of the touch screen selected by the user and transmitting an input command corresponding to the part of the touch screen selected by the user to the processor, wherein when an input command is received by the touch screen the input command is transmitted to the processor and the processor converts the input command into an output command.

51. The infusion pump of claim 48, further comprising an alarm monitor operably connected to the processor, the alarm monitor being responsive to at least one predetermined condition, wherein the at least one predetermined condition causes the alarm monitor to generate an input command which is transmitted to the processor.

52. The infusion pump system of claim 48, wherein the touch screen is also the display.
